(19) United States
(12) Patent Application Publication
(10) Pub. No.: US 2025/0034194 A1
(43) Pub. Date: Jan. 30, 2025

(54) INHIBITORS OF ABNORMAL PHOSPHORYLATION AND TAU PROTEIN AGGREGATION

(71) Applicants: UNIVERSITE DE PICARDIE JULES VERNE, AMIENS (FR); UNIVERSITE PARIS EST CRETEIL VAL DE MARNE, CRETEIL (FR); CENTRE NATIONAL DE LA RECHERCHE SCIENTIFIQUE (CNRS), PARIS (FR)

(72) Inventors: Anne WADOUACHI, Rivery (FR); Dulce PAPY GARCIA, CHILLY MAZARIN (FR); Gwladys POURCEAU, Louplande (FR); Sandrine CHANTEPIE, Draveil (FR)

(73) Assignees: UNIVERSITE DE PICARDIE JULES VERNE, AMIENS (FR); UNIVERSITE PARIS EST CRETEIL VAL DE MARNE, CRETEIL (FR); CENTRE NATIONAL DE LA RECHERCHE SCIENTIFIQUE (CNRS), PARIS (FR)

(21) Appl. No.: 18/714,018

(22) PCT Filed: Nov. 29, 2022
(86) PCT No.: PCT/EP2022/083709

§ 371 Date: May 28, 2024

(30) Foreign Application Priority Data
Nov. 29, 2021 (EP) 21306658.2

(51) Int. Cl. C07H 15/04; A61K 31/7032

(52) U.S. Cl. CPC C07H 15/04; A61K 31/7032

(57) ABSTRACT

The present disclosure relates to lipid anionic oligosaccharides and to the uses thereof in medicine, especially in the treatment of the pathologies associated with abnormal hyperphosphorylation of tau protein, such as Alzheimer's disease.

### FIELD OF THE INVENTION

The present invention concerns anionic lipid oligosaccharides and their uses in medicine, particularly in the treatment of pathologies associated with abnormal hyperphosphorylation of the tau protein, such as Alzheimer's disease.

### TECHNOLOGICAL BACKGROUND

Alzheimer's disease (AD) is the most common age-related dementia in the world. It is the cause of 60-70% of cases of dementia according to the World Health Organisation (WHO). More than 50 million people worldwide are affected by AD and 10 million new cases are recorded each year. According to WHO forecasts, the total number of people with dementia is expected to reach 82 million in 2030 and 152 million by 2050.

This disease not only has an impact on the health and morale of patients and their families, but it also constitutes a significant economic burden on society.

AD is characterized by a progressive loss of cognitive abilities related to severe neuronal degeneration. Histopathologically, AD is characterized by two types of brain lesions: on the one hand, senile plaques (or amyloids) which are extracellular aggregates of beta-amyloid peptides (Aβ) and, on the other hand, neurofibrillary tangles (NFTs) corresponding to the intracellular accumulation of abnormally phosphorylated tau protein (MAPT, microtubule-associated protein tau) (p-tau).

Despite a major effort in academic and industrial research during the last 20 years, no treatment developed has made it possible to block the progression of neurodegeneration effectively and sustainably.

As the involvement of amyloid plaques and NFTs made of p-tau in the functional alterations of neurons is well accepted, most of the treatments developed have especially targeted the progressive accumulation of the beta-amyloid protein and, more recently, of tau. However, therapeutic failures of strategies targeting different episodes of the amyloid J3 (AJ3) cascade have indicated that AJ3 accumulation is not the central pathological process in the disease, but rather it might be abnormal phosphorylation and aggregation of tau which should be targeted in order to fight against this disease.

Tau protein is a member of the microtubule-associated protein (MAP protein) family. In humans, one of the main roles of the tau protein is to stabilize the cytoskeleton of neurons via its interaction with tubulin which allows axonal stability. In AD, the abnormal phosphorylation of tau induces detachment of the protein from the cytoskeleton, and the soluble tau protein aggregates to ultimately form the NFTs characteristic of AD. A direct correlation has been shown between the clinical evolution of the disease and the accumulation of abnormally phosphorylated and aggregated tau protein in the form of neurofibrillary tangles (NFTs) in the brain.

Several kinases are capable of phosphorylating tau (cdK5, NCLK, GSK-3β and MARK), but this occurs in a non-specific manner since several other proteins are also their substrates. Thus, inhibiting one of the kinases will not prevent the action of the others. In agreement with this, and although much work has been carried out on kinase inhibitors as well as phosphatases (also non-specific) considered as therapeutic targets, no positive results making it possible to stop or slow down the disease by inhibiting the activity of these enzymes individually have been obtained to date, whether by kinase inhibitors, phosphatase activators, or NFT dissociation agents.

To date, treatments in development targeting the tau protein are mainly based on immunotherapy at the extracellular level. The antibodies used are directed against tau fragments aimed at reducing the more or less aggregated extracellular forms of the tau protein. However, these extracellular forms are generated intracellularly by the phosphorylation of the tau protein; after secretion outside the cell, these aggregated forms of tau seem to serve to propagate the pathology in the brain tissue. Currently, immunotherapies targeting the tau protein remain the main hope in the field, with all the inherent difficulties of immunotherapy, especially relating to the inaccessibility of intracellular antibodies to stop the process of abnormal phosphorylation and aggregation of tau, the stability of these products, the production of anti-antibodies, and the need for treatment in a hospital environment (Schroeder S K et al., Neuroimmune Pharmacol. 2016, 11(1):9-25.). It is therefore generally agreed that the best strategy will be the one which will involve a small, stable molecule capable of blocking the hyperphosphorylation and aggregation of tau and which can be administered to patients by a conventional regimen in a non-hospital environment.

Thus, there is still a need to provide compounds capable of stopping or slowing the progression of pathologies linked to hyperphosphorylation of the tau protein, in particular Alzheimer's disease.

### BRIEF DESCRIPTION OF THE INVENTION

The present invention relates to compounds of formula (I) below:

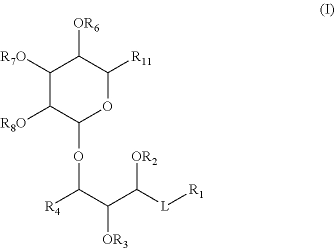

in which:
L is a —C(═O)NH—, —C(═O)O— or —C(═O)S— group;
R₁ is a linear or branched, saturated or unsaturated hydrocarbon chain, comprising 15, 16, 17 or 18 carbon atoms, optionally substituted in the terminal position by a group chosen from —OH, —OR, —NH₂, —NHR, —NRR′, —COOH, —COOR, —CONHR, —CONRR′, and —SR, with R and R′ independently designating an alkyl group comprising from one to six carbon atoms;
R₄ is a group of formula (IIa) or (IIb):

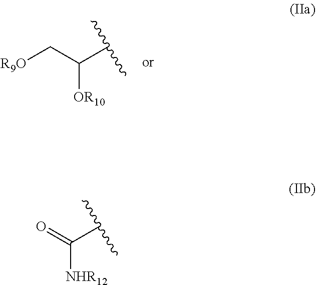

in which:
R₉ and R₁₀ are, independently of each other, a hydrogen atom or a sulphonate group in ionized form;
R₁₂ is an alkyl group comprising one to six carbon atoms;
   R₁₁ is a —CH₂—OR₅ or —CONHR₁₃ group in which:
R₅ is a hydrogen atom or a sulphonate group in ionized form;
R₁₃ is an alkyl group comprising one to six carbon atoms;
   R₂, R₃, R₅, R₇, and R₈ are, independently of each other, a hydrogen atom or a sulphonate group in ionized form; and
   in which at least one of the R₂, R₃, R₅, R₆, R₇, R₈, R₉ and R₁₀ groups, preferably at least two of the R₂, R₃, R₅, R₆, R₇, R₈, R₉ and R₁₀ groups, are a sulphonate group in ionized form, for their use in medicine, in particular in the treatment of tauopathies.

The present invention also relates to pharmaceutical compositions comprising at least one compound of formula (I) as described herein and one or more pharmaceutically acceptable excipients and their use in the treatment of tauopathies.

Other aspects of the invention are as described below and in the claims.

### FIGURES

FIG. 1: Illustration of the role of heparan sulphates (HS).

FIG. 2: Western blot showing that phosphorylation of tau at sites characteristic of Alzheimer's disease mediated by GSK3β is dependent on the presence of heparin in the reaction mixture (Sepulveda-Diaz J E et al., Brain. 2015 May; 138 (Pt 5):1339-54).

FIG. 3: Western blot showing that the abnormal phosphorylation of tau (p-tau) revealed by the antibodies characteristic of Alzheimer's disease (AT270 and Ser199/202) is inhibited by the presence of disulphated C16 maltobionamides (Mal16diS) after 3 h of in vitro reaction.

FIG. 4: Western blot showing that the abnormal phosphorylation of tau (p-tau) revealed by the antibodies characteristic of Alzheimer's disease (AT270, pSer199/202) is inhibited by the presence of disulphated C18 maltobionamides (Mal18diS) after 3 h of reaction.

FIG. 5: Western blot comparing the p-tau induction capacity (revealed by the AT270 antibody) of compounds derived from disulphated maltobionamide as a function of the length of the hydrocarbon chain (R₁) (Mal14diS to Mal20diS corresponding to chains of 14 to 20 carbons) and heparin (“Hep”) after 3 h of reaction.

FIG. 6: Western blot evaluating the abnormal phosphorylation of tau (p-tau), revealed by the antibodies characteristic of Alzheimer's disease (AT270 and Ser199/202) for the different derivatives according to their disaccharide (mono or disulphated) and the length of the hydrocarbon chain (14 to 20 carbons) after 3 h of reaction.

FIG. 7: Western blot comparing the ability to protect the tau protein from its abnormal phosphorylation induced by heparin, revealed by the AT270 antibody, of disulphated maltobionamides (Mal16diS, Mal18diS) compared to the oxidized forms with 16 carbons (Mal16diOx) and 18 carbons (Mal18diOx).

FIG. 8: Western blot comparing the effect of sulphation (monoS and triS) of the disaccharide on the capacity of 16 or 18 carbon maltobionamides to effectively protect tau from its abnormal phosphorylation induced by heparin, revealed by the AT270 antibody with 2 batches of compounds (Mal16monoS, Mal16triS for Mal16 and Mal18monoS and Mal18triS for Mal18).

FIG. 9: Western blot comparing persulphated 16-carbon maltobionamide molecules (Mal16perS) to their effective disulphated homologs (Mal16diS, Mal18diS) in their ability to effectively protect tau from its abnormal heparin-induced phosphorylation, revealed by the AT270 antibody.

FIG. 10: Monitoring the aggregation of the tau protein by integration of Thioflavin-T, a fluorescent compound which integrates into the aggregates, in the presence of heparin (tau:hep mass ratio of 2:1 or 4:1) or the 16-carbon disulphated maltobionamide compound (Mal16diS) (tau:oligo mass ratio, 4:1). The number of repetitions is represented by n.

FIG. 11: Cytotoxicity of 16-carbon disulphated maltobionamides (Mal16diS) at concentrations of 0.1, 1, 10, or 100 μg/mL.

FIG. 12: Cytotoxicity of 18-carbon disulphated maltobionamide (Mal18diS) at concentrations of 0.1, 1, 1, 10 or 100 μg/mL.

FIG. 13: Protection from tau phosphorylation in cellulo by 16-carbon disulphated maltobionamide compounds (Mal16diS) at concentrations of 1, 10 or 100 μg/mL. t-test: * p<0.05, *** p<0.001; number of replicates ≥5.

FIG. 14: Protection from tau phosphorylation in cellulo by 18-carbon disulphated maltobionamide compounds (Mal18diS) at concentrations of 1, 10 or 100 μg/ml. t-test: * p<0.05, *** p<0.001; number of replicates ≥5.

FIG. 15: Effect of Cell16diS on p-tau in cellulo in the SHSY5Y model with induction of tauopathy. t-test not significant; number of replicates ≥3.

FIG. 16: Effect of Lac16diS on p-tau in cellulo in the SHSY5Y model with induction of tauopathy. t-test: * p<0.05; number of replicates ≥3

FIG. 17: Decrease in tau phosphorylation present moderately (day 14 of culture) in cortical cells of the rTg4510 mouse model by 16-carbon disulphated maltobionamide compounds (Mal16diS) at concentrations of 0.1 or 1 μg/mL. t-test: ** p<0.01, *** p<0.001; number of replicates=4.

FIG. 18: Decrease in tau phosphorylation present severely (day 18 of culture) in cortical cells of the rTg4510 mouse model by 16-carbon disulphated maltobionamide compounds (Mal16diS) at concentrations of 0.1 or 1 μg/mL. t-test: not significant.; number of replicates=4.

### DETAILED DESCRIPTION OF THE INVENTION

It has been discovered that anionic lipid oligosaccharides as described below (compounds of formula (I)) are capable of protecting the tau protein from its abnormal hyperphosphorylation, do not themselves induce the abnormal hyperphosphorylation of tau and are capable of reducing the aggregation of the tau protein induced by heparin, the prototype of the hypersulphated heparan sulphates present in Alzheimer's disease (Sepulveda-Diaz J E et al., Brain. 2015 May; 138 (Pt 5):1339-54).

The anionic lipid oligosaccharides of the present invention therefore prove to be useful in the treatment of pathologies associated with abnormal tau protein hyperphosphorylation, and particularly in the treatment of Alzheimer's disease. The compounds may prove capable of stopping or slowing the progression of the disease.

These anionic lipid oligosaccharides mimic certain characteristics of heparan sulphates. The team of biologists at Gly-CRRET (Holmes B B et al., Proc Natl Acad Sci USA. 2013 Aug. 13; 110(33); Sepulveda-Diaz J E et al., Brain. 2015 May; 138(Pt 5):1339-54; Maïza A et al, FEBS Lett. 2018 December; 592(23):3806-3818), and other teams around the world (Goedert M et al., Nature. 1996 Oct. 10; 383(6600):550-3; Zhao J et al., Biophys J. 2017 Mar. 14; 112(5):921-932), showed that heparan sulphates are critical for the development and progression of the disease:

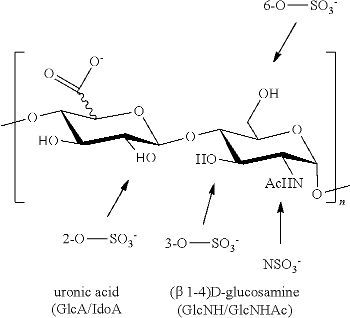

Heparan sulphates are copolymers of uronic acid (D-glucuronic or L-iduronic) and D-glucosamine which can be N-acetylated and contain sulphated sites.

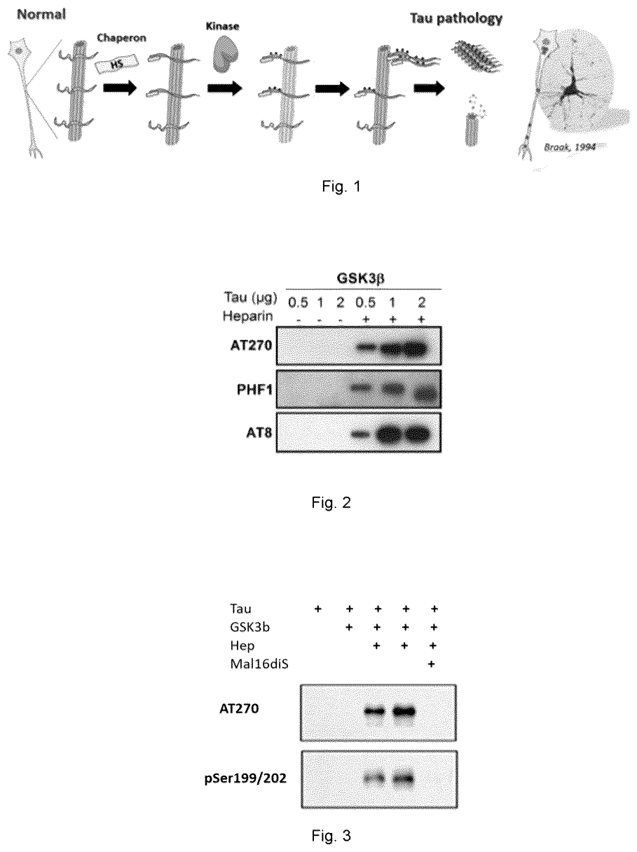

The Gly-CRRET laboratory has especially shown that the conformational change of tau allowing its abnormal hyperphosphorylation and its aggregation in neurons results from the interaction of the protein with heparan sulphate (HS) chains internalized in the neurons of subjects with the disease. By playing a role as molecular chaperones, HS allows the tau protein to acquire a conformation that favours kinase attack, which results in abnormal hyperphosphorylation and aggregation of the protein (FIG. 1).

In addition, studies carried out by the Gly-CRRET and other teams show that HS are receptors for tau aggregates (proteopathic seeds) on the surface of cells and that they centrally participate in the transfer of tau fibrils from a diseased cell to a healthy cell, a process known as spreading (Holmes B B et al., Proc Natl Acad Sci US A. 2013 Aug. 13; 110(33). tau spreading is currently considered responsible for the spread of the disease between cells and between different regions of the brain. The presence of HS in tau inclusions in vivo, as well as their capacity to trigger abnormal tau phosphorylation, aggregation and spreading, suggest a central role of these polysaccharides in the establishment and progression of the neurodegenerative process.

### Lipid Anionic Oligosaccharides

The compounds of the present invention or useful in the context of the present invention are compounds of formula (I) below:

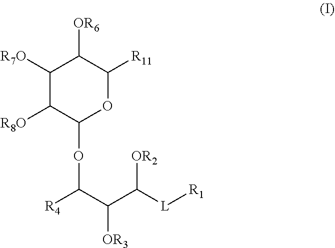

in which:
L is a —C(═O)NH—, —C(═O)O— or —C(═O)S— group;
R₁ is a linear or branched, saturated or unsaturated hydrocarbon chain, comprising 15, 16, 17 or 18 carbon atoms, optionally substituted in the terminal position by a group chosen from —OH, —OR, —NH₂, —NHR, —NRR′, —COOH, —COOR, —CONHR, —CONRR′, and —SR, with R and R′ independently designating an alkyl group comprising from one to six carbon atoms;
R₄ is a group of formula (IIa) or (IIb):

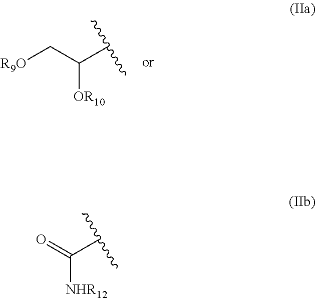

in which:
R₉ and R₁₀ are, independently of each other, a hydrogen atom or a sulphonate group in ionized form;
R₁₂ is an alkyl group comprising one to six carbon atoms;
   R₁₁ is a —CH₂—OR₅ or —CONHR₁₃ group in which:
R₅ is a hydrogen atom or a sulphonate group in ionized form;
R₁₃ is an alkyl group comprising one to six carbon atoms;
   R₂, R₃, R₆, R₇ and R₈ are, independently of each other, a hydrogen atom or a sulphonate group in ionized form; and
   in which at least one of the R₂, R₃, R₅, R₆, R₇, R₈, R₉ and R₁₀ groups, preferably at least two of the R₂, R₃, R₅, R₆, R₇, R₈, R₉ and R₁₀ groups are a sulphonate group in ionized form.

More particularly, the compounds of the present invention or useful in the context of the present invention are compounds of formula (I) below:

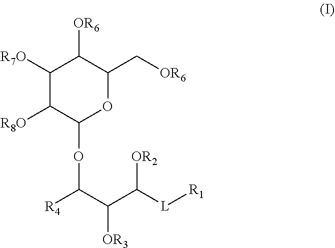

in which:
L is a —C(═O)NH—, —C(═O)O— or —C(═O)S— group;
R₁ is a linear or branched, saturated or unsaturated hydrocarbon chain, comprising 15, 16, 17 or 18 carbon atoms, optionally substituted in the terminal position by a group chosen from —OH, —OR, —NH₂, —NHR, —NRR′, —COOH, —COOR, —CONHR, —CONRR′, and —SR, with R and R′ independently designating an alkyl group comprising from one to six carbon atoms;
R₂ is a hydrogen atom or a sulphonate group in ionized form;
R₃ is a hydrogen atom or a sulphonate group in ionized form;
R₄ is a group of formula (IIa):

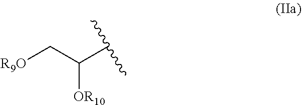

in which:
R₉ and R₁₀ are, independently of each other, a hydrogen atom or a sulphonate group in ionized form;
   R₅, R₇ and R₈ are, independently of each other, a hydrogen atom or a sulphonate group in ionized form;
   R₆ is a hydrogen atom or a sulphonate group in ionized form; and
   in which at least one of the R₂, R₃, R₅, R₆, R₇, R₈, R₉ and R₁₀ groups, preferably at least two of the R₂, R₃, R₆, R₇, R₈, R₉ and R₁₀, groups are a sulphonate group in ionized form.

The expression “sulphonate group in ionized form” designates the —SO₃— group.

The compounds of formula (I) are preferably in the form of salts, in particular in the form of sodium salts. Thus the sulphonate groups in ionized form are preferably sodium sulphonate groups (—SO₃Na).

In some embodiments, the compounds of formula (I) have two, three, four, five, six, seven or eight sulphonate groups in ionized form. Thus, two, three, four, five, six, seven or all of the R₂, R₃, R₅, R₆, R₇, R₈, R₉ and R₁₀ groups are sulphonate groups in ionized form. However, since highly charged compounds can induce toxicity, compounds comprising two or three ionized sulphonate groups may be preferred.

In some embodiments, L is a —C(═O)NH— group.

In some embodiments, the compounds of the present invention or useful in the context of the present invention have the following formula (Ia):

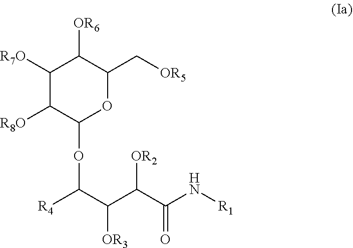

in which R₁ to R₈ are as described above and in which at least one of the R₂, R₃, R₅, R₆, R₇, R₈, R₉ and R₁₀ groups, preferably at least two of the R₂, R₃, R₅, R₆, R₇, R₈, R₉ and R₁₀ groups, are a sulphonate group in ionized form.

In some embodiments, the compounds of the present invention or useful in the context of the present invention have the following formula (Ib):

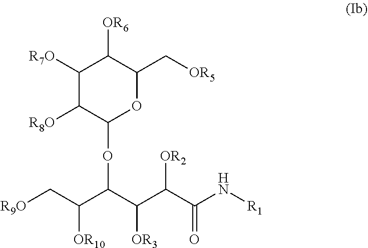

in which R₁ to R₃ and R₅ to R₁₀ are as described above or below and in which at least one of the R₂, R₃, R₅, R₆, R₇, R₈, R₉ and R₁₀ groups, preferably at least two of the R₂, R₃, R₅, R₆, R₇, R₈, R₉ and R₁₀ groups, are a sulphonate group in ionized form.

In formulas (I), (Ia) and (Ib), R₁ is preferably a saturated linear hydrocarbon chain comprising 15, 16, 17 or 18 carbon atoms, preferably 16 and 18 carbon atoms.

In formulas (I), (Ia) and (Ib), R₂, R₃ and R₁₀ are preferably a hydrogen atom.

In formulas (I), (Ia) and (Ib), R₅ is preferably a sulphonate group in ionized form and R₆, R₇, and R₈ are hydrogen atoms.

In formulas (I), (Ia) and (Ib), R₉ is preferably a sulphonate group in ionized form.

In some embodiments, the compounds of the present invention or useful in the context of the present invention have the following formula (Ib):

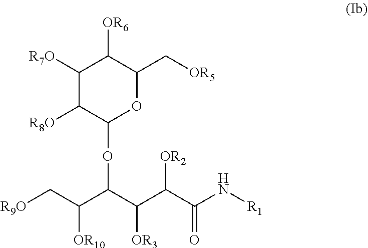

in which:
R₁ is a saturated linear hydrocarbon chain comprising 15, 16, 17 or 18 carbon atoms, preferably 16 or 18 carbon atoms;
R₂, R₃, R₆, R₇, R₈ and R₁₀ are hydrogen atoms, or only one of the R₂, R₃, R₆, R₇, R₈ and R₁₀ groups is a sulphonate group in ionized form and the other groups are hydrogen atoms; and
R₅ and R₉ are sulphonate groups in ionized form.

In some embodiments, the compounds of the present invention or useful in the context of the present invention have the following formula (Ib):

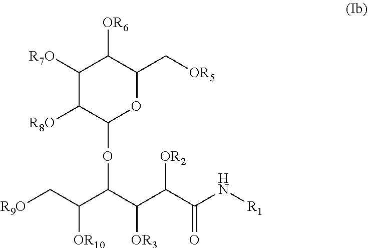

in which:
R₁ is a saturated linear hydrocarbon chain comprising 15, 16, 17 or 18 carbon atoms, preferably 16 or 18 carbon atoms;
R₂, R₃, R₆, R₇, R₈ and R₁₀ are hydrogen atoms; and
R₅ and R₉ are sulphonate groups in ionized form.

In some embodiments, the compounds of the present invention or useful in the context of the present invention have the following structure (Ic):

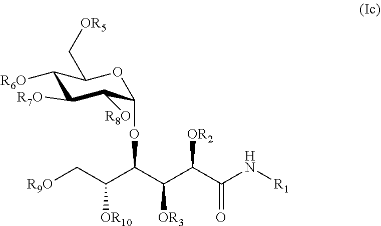

in which R₁ to R₃ and R₅ to R₁₀ are as described above.

In some embodiments, the compounds of the present invention or useful in the context of the present invention have the following structure (Id):

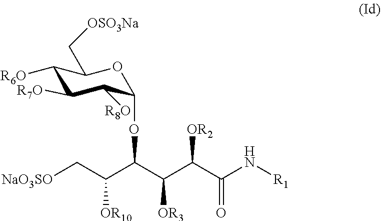

in which R₁, R₂, R₃ and R₅ to R₈ and R₁₀ are as described above.

In some embodiments, the compounds of the present invention or useful in the context of the present invention have the following structure (Ie):

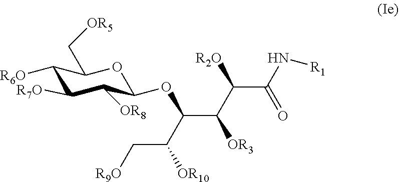

in which R₁ to R₃ and R₅ to R₁₀ are as described above.

In some embodiments, the compounds of the present invention or useful in the context of the present invention have the following structure (If):

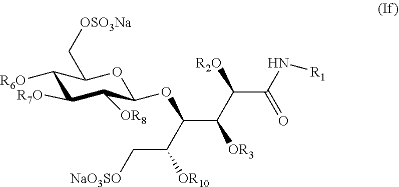

in which R₁, R₂, R₃ and R₆ to R₈ and R₁₀ are as described above.

In some embodiments, the compounds of the present invention or useful in the context of the present invention have the following structure (Ig):

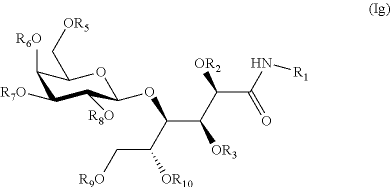

in which R₁ to R₃ and R₅ to R₁₀ are as described above.

In some embodiments, the compounds of the present invention or useful in the context of the present invention have the following structure (Ih):

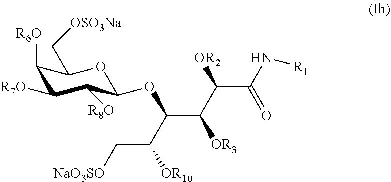

in which R₁, R₂, R₃ and R₆ to R₈ and R₁₀ are as described above.

In some embodiments, the compounds of the present invention are compounds of formula (I) provided that the compound is not sodium hexadecyl D-lactobionamide(2,5)sulphate, i.e. sodium hexadecyl D-lactobionamide disulphate, sodium hexadecyl D-lactobionamide trisulphate and mixtures thereof.

Non-limiting examples of compounds of formula (I) include compounds of formula (I) shown in Table 1 in the “Examples” section.

### Process for Preparing Compounds of Formula (I)

The diagram and procedures described below illustrate synthetic routes for the compounds of formula (I) and should not be considered limiting.

The compounds of formula (I) can be prepared in three steps from a disaccharide, such as maltose, cellobiose or lactose. The process comprises the following steps:
(step 1) oxidation of the anomeric position of the disaccharide;
(step 2) synthesis of the methyl ester and aminolysis to graft the hydrocarbon chain (R₁) under solvent-free conditions by mechanosynthesis;
(step 3) selective sulphation of primary hydroxyl groups or persulphation.

Such a process is illustrated in the reaction diagram below (Diagram 1) in which the disaccharide is maltose. It is understood that maltose can be replaced by any appropriate disaccharide.

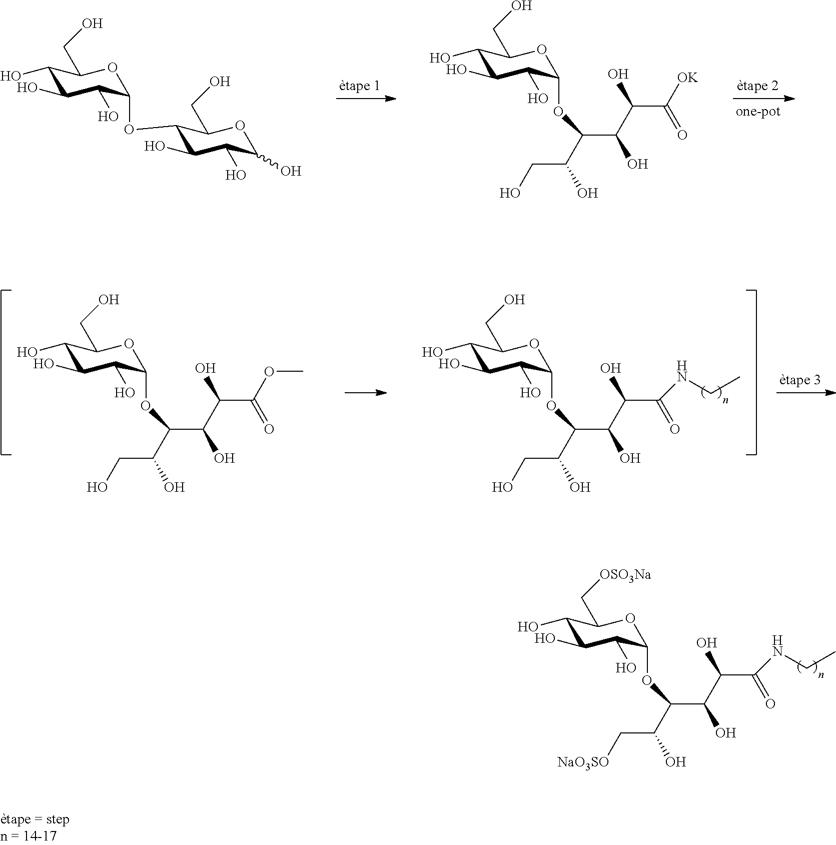

Diagram 1: synthetic route of compounds of formula (I) from maltose

### Step 1:

The anomeric position of the disaccharide can be oxidized according to well-known methods, such as, for example, by method A described in ACS Sustainable Chemistry & Engineering 2016, 4(4), 2432-2438.

Alternatively, sodium maltobionate can be prepared by photocatalysis (Omri M. et al., ACS Catalysis 2018) with the same conversion rate: >99%, selectivity: >95% (estimated by NMR).

### Step 2: One-Pot Synthesis of the N-Alkyl D-Maltobionamide Derivative

It is produced by a solvent-free mechanosynthesis process. Potassium glycobionate is ground in the presence of a supported acid catalyst (H₂SO₄/SiO₂) and methanol (in the exact quantity necessary to methylate the carboxylic acid formed) in a ball mill, then in a second step with the alkylamine added after total conversion of the glycobionate into ester. Quantitative conversion rates for step 2, typically greater than 85%, can be obtained for both steps together.

Step 3: The sulphation step is carried out according to a protocol of the literature in a pyridine medium in the presence of a sulphating agent, SO₃-pyridine (Tetrahedron. 2010 Apr. 17; 66(16): 2907-2918).

Hereinafter, the expression “a compound of formula (I)” means a compound of formula (I) as described above, including the compounds described in the “Examples” section. The expression “a compound of formula (I)” means a single compound of formula (I) or a mixture of two or more compounds of formula (I).

### Therapeutic Applications

Compounds of formula (I) have been shown to effectively protect tau protein from its abnormal heparin-induced hyperphosphorylation, weakly induce tau protein phosphorylation and reduce heparin-induced aggregation of tau protein. The compounds of formula (I) therefore prove to be useful in medicine, particularly in the treatment of pathologies linked to abnormal hyperphosphorylation or phosphorylation of the tau protein (tauopathy), and particularly in the treatment of Alzheimer's disease. The compounds of formula (I) would work by inhibiting the abnormal phosphorylation of tau, not by inhibiting kinases but by protecting tau from their attack, before the process of aggregation and propagation of aggregates.

Thus, the present invention relates to a compound of formula (I) for its use in medicine or, formulated differently, as a medicament.

More specifically, the present invention relates to a compound of formula (I) for its use in the treatment of pathologies linked to abnormal hyperphosphorylation or phosphorylation of the tau protein, commonly referred to as “tauopathies”. Non-limiting examples of tauopathies include Alzheimer's disease, frontotemporal dementia with parkinsonism on chromosome 17 (FTDP-17), Pick's disease, corticobasal degeneration and progressive supranuclear palsy (PSP).

Thus, in particular embodiments, the present invention relates to a compound of formula (I) for its use in the treatment of Alzheimer's disease, frontotemporal dementia with parkinsonism on chromosome 17 (FTDP-17), Pick's syndrome, corticobasal degeneration or progressive supranuclear palsy. In particular embodiments, the present invention relates to a compound of formula (I) for use in the treatment of Alzheimer's disease.

The present invention also relates to the use of compound of formula (I) for the preparation of a medicament useful in the treatment of pathologies linked to abnormal hyperphosphorylation or phosphorylation of the tau protein (tauopathy), and very particularly in Alzheimer's treatment.

### Pharmaceutical Compositions

The present invention also relates to a pharmaceutical composition, in particular a medicament, comprising a compound of formula (I) and one or more excipients, in particular one or more pharmaceutically acceptable excipients and their uses in the therapeutic applications described above.

The term “pharmaceutically acceptable” refers to something which is known to be non-toxic and can be used in medicine. Pharmaceutically acceptable excipients are well known in the medical literature.

The excipients are chosen according to the desired pharmaceutical form and method of administration. Such excipients are well known to the skilled person.

The pharmaceutical compositions according to the invention can be administered parenterally, such as intravenously or intradermally, or topically, orally or nasally.

Forms that can be administered parentally include aqueous suspensions, isotonic saline solutions or sterile, injectable solutions which may contain pharmacologically compatible dispersing agents and/or wetting agents. Forms that can be administered orally include tablets which may be dispersible, orodispersible, effervescent or soluble, soft or hard capsules, powders, granules and oral solutions and suspensions. Forms that can be administered nasally include aerosols. Forms that can be administered topically include patches, gels, creams, ointments, lotions, sprays and eye drops. Preferably, the compounds or compositions of the invention are administered orally or parenterally (especially intravenously).

The present invention also concerns a method of treating the pathologies indicated above which comprises the administration, to an individual in need, of an effective dose of a compound of formula (I) or of a pharmaceutical composition comprising a compound of formula (I) and one or more pharmaceutically acceptable excipients.

The effective dose of a compound of formula (I) varies depending on numerous parameters such as, for example, the chosen route of administration, weight, age, sex, the progression of the pathology to be treated and the sensitivity of the individual to be treated.

The examples which follow are given for illustrative purposes, but should in no way be considered as limiting the present invention.

### EXAMPLES

### I. Compounds Tested

The compounds tested are presented in Table 1 below.

TABLE 1
compounds tested

| | | Structure | | Code |
|---|---|---|---|---|
| Compounds of formula (I) | Monosulphated maltobionamide | 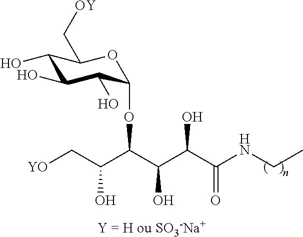 | n = 15 n = 17 | Mal 16monoS Mal 18monoS |
| | Disulphated maltobionamide | 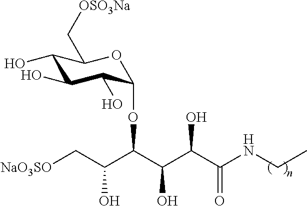 | n = 15 n = 17 | Mal 16diS Mal 18diS |
| | Trisulphated maltobionamide | 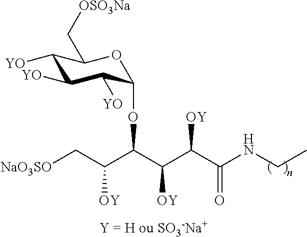 | n = 15 n = 17 | Mal 16triS Mal 18triS |
| | Persulphated maltobionamide | 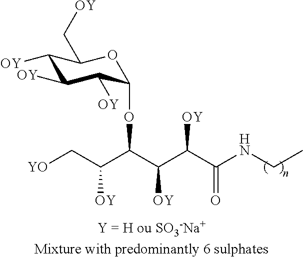 | n = 15 | Mal 16perS |
| | Disulphated cellobionamide | 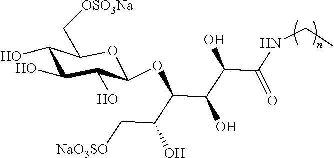 | n = 15 n = 17 | Cell 16diS Cell 18diS |
| | Disulphated lactobionamide | 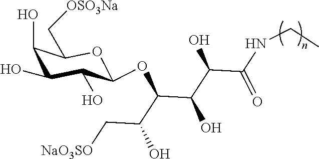 | n = 15 n = 17 | Lac 16diS Lac 18diS |
| | Persulphated maltobionamide dibutylamide | 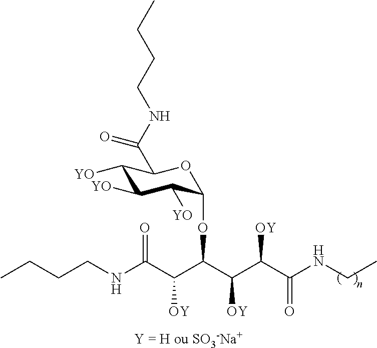 | n = 15 | Mal 16diBu- perS |
| Compounds not corresponding to formula (I) | Disulphated maltobionamide | 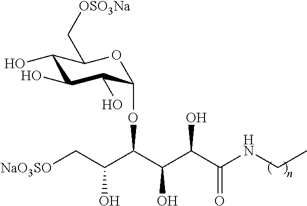 | n = 13 n = 18 n = 19 | Mal 14diS Mal 19diS Mal 20diS |
| | Dioxidized maltobionamide | 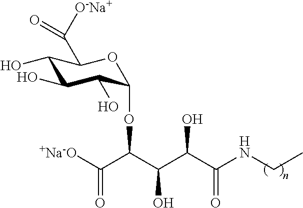 | n = 15 n = 17 | Mal 16diOx Mal 18diOx |
| | Disulphated cellobionamide | 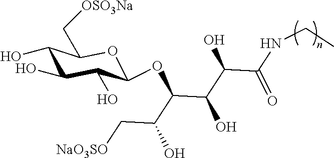 | n = 13 | Cell 14diS |
| | Disulphated lactobionamide | 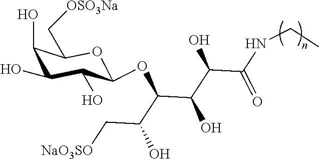 | n = 13 | Lac 14diS |
| | Monosulphated melibionamide | 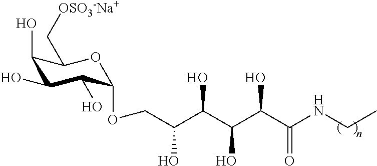 | n = 13 n = 15 n = 17 | Mel 14monoS Mel 16monoS Mel 18monoS |

ou = or

### II. Synthesis of Tested Compounds

### II-1. Chemical Materials and Methods

### a) Reagents and Solvents

The reagents used originate from Merck, Acros or Alfa Aesar and are used without purification. The solvents are purchased either in anhydrous form or are distilled under an inert atmosphere and conditioned on a 3 or 4-angstrom molecular sieve.

### b) Ball Mill

The reactions are carried out in a Fritsch Pulverisette Premium 7 (P7PL) planetary mill equipped with two 20 mL bowls each comprising 80 zirconia (ZrO₂) balls with a diameter of 5 mm.

### c) Chromatography

Thin Layer Chromatography (TLC). Thin layer chromatography was performed on silica plates with ALUGRAM® Xtra SIL G/UV₂₅₄ aluminium support for the normal phase or ALUGRAM® RP-18/UV₂₅₄ for the reversed phase. The techniques used to reveal the products after elution are: H₂SO₄/EtOH at 4% or cerium (IV) molybdate: (NH₄)₄Ce(SO₄)·2H₂O, (NH₄)₆Mo₇O₂₄·4H₂O, H₂SO₄, H₂O (2.1 g/5.3 g/12 mL/188 mL) then heating.

Automated flash chromatography. Automated flash purifications were performed with a Grace flash device (Reveleris® iES Flash System). The separation is carried out using commercially available prepacked normal or C18 silica columns (4 g, 12 g, 24 g, 40 g, 80 g). It is possible to carry out purifications in normal phase and also in reversed phase. The mass of crude products corresponds to a maximum of 10% of silica from the prepacked column. The device is equipped with a light diffusion detector (LDD) and a UV detector with two configurable wavelengths.

### d) Characterization of the Compounds

### Specific Rotatory Power

The specific rotatory powers were measured at a temperature of 20° C. using the Perkin-Elmer 343 polarimeter which emits polarized light of λ=549 nm (sodium D-line). The concentration c is expressed in grams per 100 mL of solvent.

### Low Resolution Mass Spectrometry.

Low resolution mass spectrometry analyses were carried out on a simple quadrupole device (Micromass-Waters ZQ) with an electrospray ionization source (Z-spray). The device allows analysis in positive or negative ionization mode. The capillary voltage is 3.5 kV and the cone voltage varies from 20 to 120 V. The source and desolvation temperatures are 80 and 150° C., respectively. The desolvation and nebulization gas is nitrogen.

### High Resolution Mass Spectrometry.

High-resolution mass spectrometry (HRMS) analyses were carried out on a hybrid quadrupole time-of-flight mass spectrometer (Micromass-Waters Q-TOF Ultima Global), equipped with an electrospray ionization source. The source and desolvation temperatures are 80 and 120° C., respectively. The gas used for nebulization and desolvation is nitrogen, with a respective flow rate of 20 and 500 L/h. The capillary voltage is 3.5 kV and the cone voltage varies from 100 to 250 V. Before any exact mass measurement, a calibration is carried out with orthophosphoric acid. Since the precision of the exact mass measurement of Q-TOF is less than 5 ppm, access to the elemental composition of molecules is possible.

NMR spectrometry. ¹H and ¹³C NMR spectra were produced on a Bruker Advance 400 or 600 MHz spectrometer. The ¹H NMR spectra are recorded at 400 MHz or 600 MHz and the ¹³C NMR spectra at 101 MHz or 150 MHz. All the experiments are carried out at a temperature close to 25° C., in a deuterated solvent which also serves as a reference. Chemical shifts are given in ppm and coupling constants in Hertz. The NMR spectra are processed using MestReNova.

### Carbon Numbering:

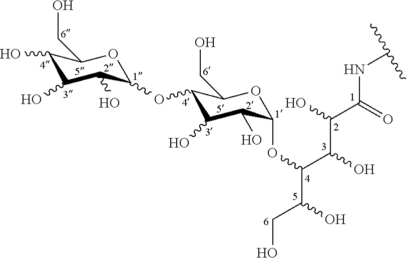

### II-2 Synthesis of Maltobionamide Sulphates

The maltobionamide sulphates, including the compounds of formula (I), were prepared according to diagram (1) presented in the description, unless otherwise indicated.

### II.2 a). Procedure for the Synthesis of Potassium Glycobionates

An aqueous solution of 5% disaccharide (250 mg in 5 mL of MilliQ water) is placed in a 10 mL CEM microwave tube, then 2 equivalents of K₂CO₃ and 2.5 mg of Au/Al₂O₃ catalyst prepared according to the protocol described in ACS Sustainable Chem. Eng., 2016, 4, 2432-2438, are added, and the mixture is subjected to ultrasound in order to homogenize it. Then 1.8 equivalents of H₂O₂ at 30% in water are added then the mixture is placed in a CEM Discover microwave and irradiated at 60° C., using the Dynamic program for a duration of 20 min. The catalyst is then filtered through 0.25 m PVDF or removed by centrifugation. The filtrate is then freeze dried and the crude reaction is analysed by ¹H NMR in order to monitor the total conversion of aldose to aldonate. The reaction can be carried out on 1 g of disaccharide in a 35 mL tube with a reaction time of 50 min to 70 min depending on the disaccharide.

### Potassium D-Maltobionate (Mal)

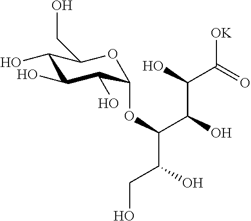

### Quantitative Yield

Appearance: solid white
Molecular formula: C₁₂H₂₁KO₁₂
MM: 396.39 g·mol⁻¹
ESI-MS: [M−K]⁻ m/z 357 Da

¹H NMR (400 MHz, D₂O): δ5.20 (d, J=3.9 Hz, 1H, H-1′), 4.17 (dd, J=6.2, 2.6 Hz, 1H, H-3), 4.13 (d, J=2.7 Hz, 1H, H-2), 4.03 (dt, J=7.7, 3.7 Hz, 1H, H-5), 3.98-3.89 (m, 2H, H-5′, H-4), 3.89-3.80 (m, 3H, 1H-6′a, H-6′b, H-6a), 3.77 (dd, J=9.2, 9.9 Hz, 1H, H-3′), 3.71 (dd, 1H, J=11.9, 7.8 Hz, H-6b), 3.59 (dd, J=9.9, 3.8 Hz, 1H, H-2′), 3.47 (dd, J=9.1, 10.1 Hz, 1H, H-4′). ¹³C NMR (101 MHz, D₂O): δ178.2 (C-1), 100.4 (C-1′), 82.4 (C-4), 73.0 (C-3′), 72.6 (C-2), 72.5 (C-3), 72.4 (C-5), 72.3 (C-5′), 71.8 (C-2′), 69.3 (C-4′), 62.1 (C-6), 60.4 (C-6′).

### Potassium D-Cellobionate (Cell)

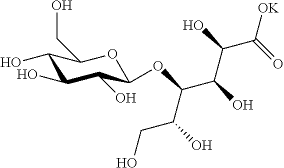

### Quantitative Yield

Appearance: solid white
Molecular formula: C₁₂1H₂₁KO₁₂
MM: 396.39 g·mol⁻¹
ESI-MS: [M−K]⁻ m/z 357 Da

¹H NMR (400 MHz, D₂O): δ4.63 (d, J=7.9 Hz, 1H, H-1′), 4.16 (d, J=3.0 Hz, 1H, H-2), 4.09 (dd, J=5.4, 3.0 Hz, 1H, H-3), 4.05-3.94 (m, 2H, H-4, H-5), 3.91 (dd, J=12.4, 2.1 Hz, 1H, H-6′a), 3.85 (dd, J=12.0, 3.1 Hz, 1H, H-6a), 3.78-3.71 (m, 2H, H-6b, H-6′b), 3.52 (t, J=9.0 Hz, 1H, H-3′), 3.49-3.44 (m, 1H, H-5′), 3.44-3.38 (m, 1H, H-4′), 3.35 (dd, J=9.4, 7.9 Hz, 1H, H-2′). ¹³C NMR (101 MHz, D₂O): δ178.4 (C-1), 103.0 (C-1′), 81.8 (C-4), 76.0 (C-5′), 75.6 (C-3′), 73.4 (C-2′), 72.4 (C-2), 71.8 (C-5), 71.6 (C-3), 69.5 (C-4′), 61.9 (C-6), 60.6 (C-6′).

### Potassium D-Lactobionate (Lac)

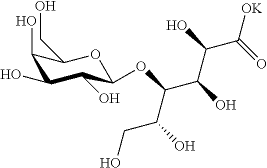

### Quantitative Yield

Appearance: solid white
Molecular formula: C₁₂H₂₁KO₁₂
MM: 396.39 g·mol⁻¹
ESI-MS: [M−K]⁻ m/z 357 Da

¹H NMR (400 MHz, D₂O): δ4.59 (d, J=7.8 Hz, 1H, H-1′), 4.20 (d, J=2.9 Hz, 1H, H-2), 4.13 (dd, J=5.3, 2.9 Hz, 1H, H-3), 4.06-3.97 (m, 2H, H-4, H-5), 3.94 (d, J=3.4 Hz, 1H, H-4′), 3.88 (dd, J=12.0, 3.1 Hz, 1H, H-6a), 3.84-3.72 (m, 4H, H-6′a, H-6b, H-6′b, H-5′), 3.70 (dd, J=10.0, 3.4 Hz, 1H, H-3′), 3.59 (dd, J=10.0, 7.8 Hz, 1H, H-2′). ¹¹C NMR (101 MHz, D₂O): δ178.5 (C-1), 103.5 (C-1′), 81.7 (C-4), 75.3 (C-5′), 72.6 (C-3′), 72.5 (C-2), 71.8 (C-5), 71.6 (C-3), 71.1 (C-2′), 68.7 (C-4′), 61.9 (C-6), 61.1 (C-6′).

II.2 b). Protocol for synthesis of N-alkyl-D-glycobionamide via methyl D-glycobionate One gram of crude reactional potassium glycobionate and 1.1 eq (˜1 g) of sulphuric acid supported on commercial 22% silica (H₂SO₄/SiO₂) are placed in a 20 mL zirconia bowl equipped with 80 zirconia (ZrO₂) balls with a diameter of 5 mm. One millilitre of methanol is added and the mixture is ground at 500 rpm for 8 cycles of 5 min in the P7PL. The reaction is then monitored by thin layer chromatography (TLC) in normal phase (eluent: ethyl acetate/methanol/water 7/2/1). The amine (1.5 eq.) is then added to the reaction medium in the bowl, followed by 2 mL of methanol. Milling is continued for 12×5 min at 500 rpm. The reaction is again monitored by TLC with the same eluent. Once the reaction is complete, the reaction medium is left to dry at room temperature. This reground powder is then mixed with unground silica and subjected to purification by flash chromatography using the same eluent as for monitoring the reaction. The fraction containing glycobionamide is concentrated in a rotary evaporator before being freeze-dried.

### N-Tetradecyl-D-Maltobionamide (Mal14)

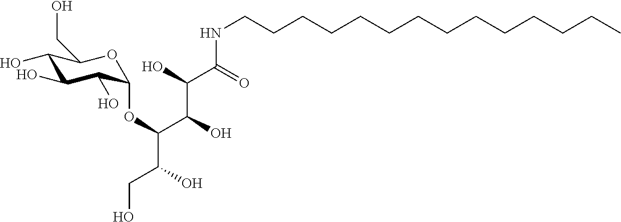

Yield: 93%
Appearance: solid white
Molecular formula: C₂₆H₅₁NO₁₁
MM: 553.68 g·mol⁻¹

¹H NMR (400 MHz, pyridine-d₅): δ8.23 (t, 1H, J=6.0 Hz, NH), 5.76 (d, 1H, J=3.9 Hz, H-1′), 5.25 (dd, 1H, J=2.0 Hz, J=5.3 Hz, H-3), 5.12 (d, 1H, J=1.8 Hz, H-2), 4.76 (t, 1H, J=5.0 Hz, H-4), 4.74-4.67 (m, 2H, H-5, H-5′), 4.60 (t, 1H, J=9.2 Hz, H-3′), 4.54 (dd, 1H, J=1.9 Hz, J=11.5 Hz, H-6′a), 4.40 (dd, 1H, J=4.7 Hz, J=11.3 Hz, H-6a), 4.37-4.28 (m, 2H, H-6b, H-6′b), 4.19 (dd, 1H, J=3.9 Hz, J=9.5 Hz, H-2′), 4.15 (t, 1H, J=9.5 Hz, H-4′), 3.55-3.40 (m, 2H, CH₂—NH), 1.54 (p, 2H, J=7.2 Hz, CH₂—CH₂—NH), 1.33-1.12 (m, 22H, CH₂ alkyl chain), 0.88 (t, 3H, J=6.6 Hz, CH₃)

¹³C NMR (101 MHz, pyridine-d₅): δ174.0 (C-1), 103.4 (C-1′), 85.5 (C-4), 75.9 (C-3′), 75.6 (C-5′), 74.8 (C-5), 74.4 (C-2′), 74.3 (C-2), 74.1 (C-3), 72.4 (C-4′), 64.8 (C-6), 63.1 (C-6′), 39.9 (CH₂—NH), 32.6-23.4 (CH₂ alkyl chain), 14.8 (CH₃).

### N-Hexadecyl-D-Maltobionamide (Mal16)

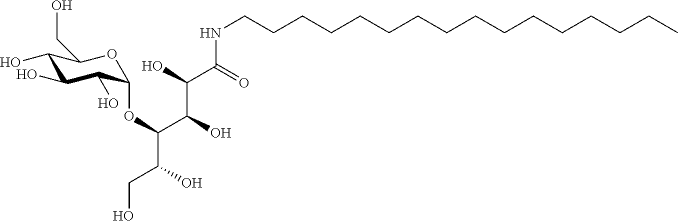

Yield: 71%
Appearance: solid white
Molecular formula: C₂₈H₅₅NO₁₁
MM: 581.74 g·mol⁻¹
Yield: 0.72 (EtOAc/MeOH/H₂O: 7/2/1)
[α]_{D}²⁰: +71.4° (MeOH, c=0.5)
ESI-HRMS: [M+Na]⁺604.3663 (calculated: 604.3673 for C₂₈H₅₅NO₁₁Na)

¹H NMR (600 MHz, pyridine-d₅): δ8.25 (t, 1H, J=5.3 Hz, NH), 5.78 (d, 1H, J=3.9 Hz, H-1′), 5.25 (dd, 1H, J=2.0 Hz, J=5.3 Hz, H-3), 5.13 (d, 1H, J=1.8 Hz, H-2), 4.77 (t, 1H, J=5.0 Hz, H-4), 4.73-4.69 (m, 2H, H-5, H-5′), 4.59 (t, 1H, J=9.2 Hz, H-3′), 4.53 (dd, 1H, J=1.9 Hz, J=11.5 Hz, H-6′a), 4.38 (dd, 1H, J=4.7 Hz, J=11.3 Hz, H-6a), 4.34-4.29 (m, 2H, H-6b, H-6′b), 4.18 (dd, 1H, J=3.9 Hz, J=9.5 Hz, H-2′), 4.14 (t, 1H, J=9.5 Hz, H-4′), 3.52-3.40 (m, 2H, CH₂—NH), 1.53 (p, 2H, J=7.2 Hz, CH₂—CH₂—NH), 1.29-1.16 (m, 26H, CH₂ alkyl chain), 0.87 (t, 3H, J=6.6 Hz, CH₃)

¹³C NMR (151 MHz, pyridine-d₅): δ173.8 (C-1), 103.3 (C-1′), 85.3 (C-4), 75.8 (C-3′), 75.5 (C-5′), 74.7 (C-5), 74.3 (C-2′), 74.2 (C-2), 74.0 (C-3), 72.3 (C-4′), 64.6 (C-6), 63.0 (C-6′), 39.8 (CH₂—NH), 32.5-23.3 (CH₂ alkyl chain), 14.6 (CH₃).

### N-Octadecyl-D-Maltobionamide (Mal18)

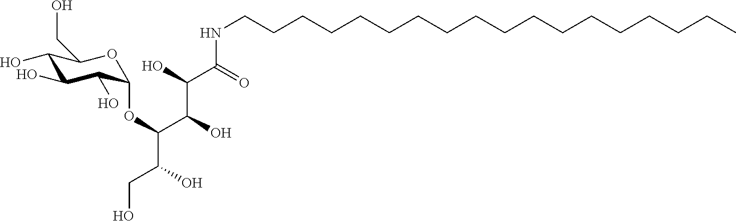

Yield: 87%
Appearance: solid white
Molecular formula: C₃₀H₅₉NO₁₁
MM: 609.80 g·mol⁻¹
Yield: 0.71 (EtOAc/MeOH/H₂O: 7/2/1).
[α]_{D}²⁰: +70.8 (MeOH, c=0.5)
ESI-HRMS: [M+Na]⁺632.3980 (calculated: 632.3986 for C₃₀H₅₉NO₁₁Na)

¹H NMR (600 MHz, pyridine-d₅): δ8.23 (t, 1H, J=5.5 Hz, NH), 5.77 (d, 1H, J=4.0 Hz, H-1′), 5.26 (dd, 1H, J=2.2 Hz, J=5.6 Hz, H-3), 5.13 (d, 1H, J=2.1 Hz, H-2), 4.77 (t, 1H, J=4.9 Hz, H-4), 4.74-4.69 (m, 2H, H-5, H-5′), 4.60 (t, 1H, J=9.2 Hz, H-3′), 4.53 (dd, 1H, J=2.2 Hz, J=11.6 Hz, H-6′a), 4.39 (dd, 1H, J=4.8 Hz, J=11.3 Hz, H-6a), 4.35-4.30 (m, 2H, H-6b, H-6′b), 4.18 (dd, 1H, J=4.0 Hz, J=9.5 Hz, H-2′), 4.15 (t, 1H, J=9.5 Hz, H-4′), 3.52-3.41 (m, 2H, CH₂—NH), 1.56-1.51 (m, 2H, CH₂—CH₂—NH), 1.29-1.17 (m, 30H, CH₂ alkyl chain), 0.87 (t, 3H, J=6.8 Hz, CH₃)

¹³C NMR (151 MHz, pyridine-d₅): δ173.8 (C-1), 103.3 (C-1′), 85.4 (C-4), 75.8 (C-3′), 75.5 (C-5′), 74.7 (C-5), 74.3 (C-2′), 74.2 (C-2), 74.0 (C-3), 72.3 (C-4′), 64.6 (C-6), 63.0 (C-6′), 39.8 (CH₂—NH), 32.5-23.3 (CH₂ alkyl chain), 14.6 (CH₃).

### N-Nonadecyl-D-Maltobionamide (Mal19)

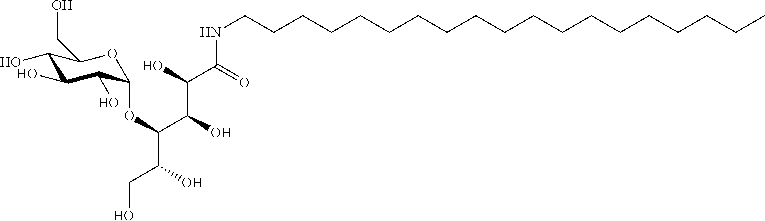

Yield: 69%
Appearance: solid white
Molecular formula: C₃₁1H₆₁NO₁₁
MM: 623.82 g·mol⁻¹

¹H NMR (400 MHz, pyridine-d₅): δ8.22 (t, 1H, J=5.5 Hz, NH), 5.78 (d, 1H, J=4.0 Hz, H-1′), 5.25 (dd, 1H, J=2.2 Hz, J=5.6 Hz, H-3), 5.13 (d, 1H, J=2.1 Hz, H-2), 4.77 (t, 1H, J=4.9 Hz, H-4), 4.74-4.67 (m, 2H, H-5, H-5′), 4.60 (t, 1H, J=9.2 Hz, H-3′), 4.55 (dd, 1H, J=2.2 Hz, J=11.6 Hz, H-6′a), 4.40 (dd, 1H, J=4.8 Hz, J=11.3 Hz, H-6a), 4.37-4.28 (m, 2H, H-6b, H-6′b), 4.20 (dd, 1H, J=4.0 Hz, J=9.5 Hz, H-2′), 4.15 (t, 1H, J=9.5 Hz, H-4′), 3.55-3.40 (m, 2H, CH₂—NH), 1.58-1.51 (m, 2H, CH₂—CH₂—NH), 1.35-1.14 (m, 30H, CH₂ alkyl chain), 0.88 (t, 3H, J=6.8 Hz, CH₃)

¹³C NMR (101 MHz, pyridine-d₅): δ173.9 (C-1), 103.4 (C-1′), 85.5 (C-4), 75.9 (C-3′), 75.6 (C-5′), 74.8 (C-5), 74.4 (C-2′), 74.3 (C-2), 74.1 (C-3), 72.4 (C-4′), 64.7 (C-6), 63.1 (C-6′), 39.9 (CH₂—NH), 32.6-23.1 (CH₂ alkyl chain), 14.5 (CH₃).

### N-Eicosanyl-D-Maltobionamide (Mal20)

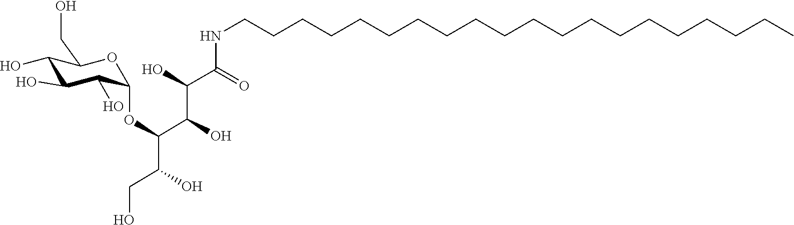

Yield: 38%
Appearance: solid white
Molecular formula: C₃₂H₆₃NO₁₁
MM: 637.84 g·mol⁻¹

¹H NMR (400 MHz, pyridine-d₅): δ8.25 (t, 1H, J=5.5 Hz, NH), 5.78 (d, 1H, J=4.0 Hz, H-1′), 5.25 (dd, 1H, J=2.2 Hz, J=5.6 Hz, H-3), 5.13 (d, 1H, J=2.1 Hz, H-2), 4.79 (t, 1H, J=4.9 Hz, H-4), 4.76-4.68 (m, 2H, H-5, H-5′), 4.61 (t, 1H, J=9.2 Hz, H-3′), 4.56 (dd, 1H, J=2.2 Hz, J=11.6 Hz, H-6′a), 4.38 (dd, 1H, J=4.8 Hz, J=11.3 Hz, H-6a), 4.44-4.27 (m, 2H, H-6b, H-6′b), 4.19 (dd, 1H, J=4.0 Hz, J=9.5 Hz, H-2′), 4.15 (t, 1H, J=9.5 Hz, H-4′), 3.55-3.40 (m, 2H, CH₂—NH), 1.59-1.50 (m, 2H, CH₂—CH₂—NH), 1.37-1.11 (m, 30H, CH₂ alkyl chain), 0.88 (t, 3H, J=6.8 Hz, CH₃)

¹³C NMR (101 MHz, pyridine-d₅): δ173.9 (C-1), 103.4 (C-1′), 85.4 (C-4), 75.9 (C-3′), 75.6 (C-5′), 74.8 (C-5), 74.4 (C-2′), 74.3 (C-2), 74.1 (C-3), 72.4 (C-4′), 64.7 (C-6), 63.1 (C-6′), 39.9 (CH₂—NH), 32.4-23.4 (CH₂ alkyl chain), 14.7 (CH₃).

### N-Tetradecyl-D-Cellobionamide (Cell14)

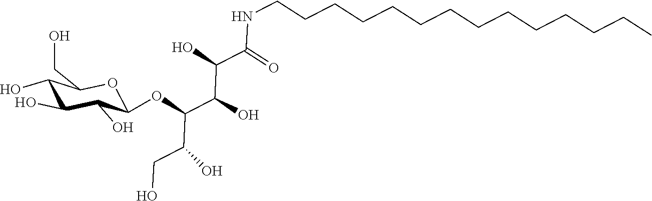

Yield: 74%
Appearance: solid white
Molecular formula: C₂₆H₅₁NO₁₁
MM: 553.68 g·mol⁻¹

¹H NMR (400 MHz, pyridine-d₅): δ8.31 (t, 1H, J=5.9 Hz, NH), 5.35 (t, 1H, J=3.2 Hz, H-3), 5.29 (d, 1H, J=2.9 Hz, H-2), 5.27 (d, 1H, J=7.9 Hz, H-1′), 4.83-4.79 (m, 2H, H-4, H-5), 4.53-4.47 (m, 3H, H-6a, H-6b, H-6a′), 4.23 (dd, 1H, J=6.7 Hz, J=11.8 Hz, H-6b′), 4.19 (t, 1H, J=8.9 Hz, H-3′), 4.06 (t, J=8.7 Hz, 2H, H-2′, H-4), 4.01-3.96 (m, 1H, H-5′), 3.53-3.37 (m, 2H, CH₂—NH), 1.56-1.50 (m, 2H, CH₂—CH₂—NH), 1.28-1.09 (m, 22H, CH₂ alkyl chain), 0.88 (t, 3H, J=6.8 Hz, CH₃)

¹³C NMR (101 MHz, pyridine-d₅): δ174.8 (C-1), 106.5 (C-1′), 84.3 (C-4), 79.2 (C-5′), 78.9 (C-3′), 76.0 (C-2′), 73.9 (C-5), 73.4 (C-2), 73.3 (C-3), 72.2 (C-4′), 65.2 (C-6), 63.4 (C-6′), 40.0 (CH₂—NH), 32.6-23.4 (CH₂ alkyl chain), 14.6 (CH₃).

### N-Hexadecyl-D-Cellobionamide (Cell16)

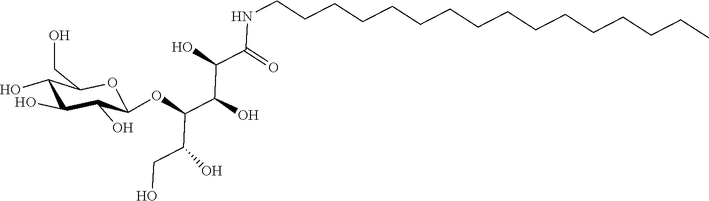

Yield: 92%
Appearance: solid white
Molecular formula: C₂₈H₅₅NO₁₁
MM: 581.74 g·mol⁻¹
Yield: 0.72 (EtOAc/MeOH/H₂O: 7/2/1)
[α]_{D}²⁰: +13.8° (MeOH, c=0.5)
ESI-HRMS: [M+H]⁺ 582.3861 (calculated: 582.3853 for C₂₈H₅₅NO₁₁)

¹H NMR (600 MHz, pyridine-d₅): δ8.30 (t, 1H, J=5.9 Hz, NH), 5.34 (t, 1H, J=3.2 Hz, H-3), 5.27 (d, 1H, J=2.9 Hz, H-2), 5.26 (d, 1H, J=7.9 Hz, H-1′), 4.83-4.79 (m, 2H, H-4, H-5), 4.53-4.47 (m, 3H, H-6a, H-6b, H-6a′), 4.22 (dd, 1H, J=6.7 Hz, J=11.8 Hz, H-6b′), 4.19 (t, 1H, J=8.9 Hz, H-3′), 4.04 (t, J=8.7 Hz, 2H, H-2′, H-4), 3.99-3.94 (m, 1H, H-5′), 3.49-3.38 (m, 2H, CH₂—NH), 1.55-1.48 (m, 2H, CH₂—CH₂—NH), 1.27-1.15 (m, 26H, CH₂ alkyl chain), 0.87 (t, 3H, J=6.8 Hz, CH₃)

¹³C NMR (151 MHz, pyridine-d₅): δ174.7 (C-1), 106.4 (C-1′), 84.2 (C-4), 79.1 (C-5′), 78.8 (C-3′), 75.9 (C-2′), 73.8 (C-5), 73.3 (C-2), 73.2 (C-3), 72.1 (C-4′), 65.1 (C-6), 63.3 (C-6′), 39.9 (CH₂—NH), 32.5-23.3 (CH₂ alkyl chain), 14.6 (CH₃).

### N-Octadecyl-D-Cellobionamide (Cell18)

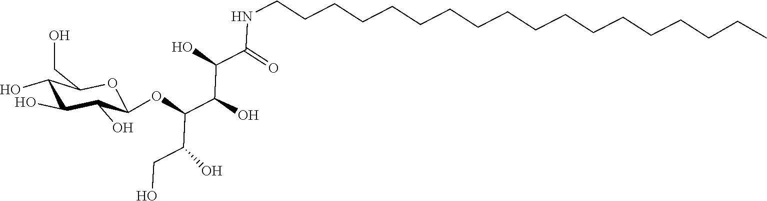

Yield: 97%
Appearance: solid white
Molecular formula: C₃₀H₅₉NO₁₁
MM: 609.41 g·mol⁻¹
Yield: 0.75 (EtOAc/MeOH/H₂O: 7/2/1)
[α]_{D}²⁰: +14.8° (MeOH, c=0.5)
ESI-HRMS: [M+H]⁺ 610.4175 (calculated: 610.4166 for C₃₀H₆₀NO₁₁)

¹H NMR (400 MHz, pyridine-d₅): δ8.31 (t, J=5.7 Hz, 1H, NH), 5.34 (br s, 1H, H-3), 5.30-5.23 (m, 2H, H-2, H-1′), 4.84-4.78 (m, 2H, H-4, H-5), 4.57-4.45 (m, 3H, H-6a, H-6b, H-6a′), 4.23 (dd, 1H, J=6.4 Hz, J=11.8 Hz, H-6b′), 4.19 (t, 1H, J=8.8 Hz, H-3′), 4.04 (t, J=8.3 Hz, 2H, H-2′, H-4′), 3.99-3.95 (m, 1H, H-5′), 3.52-3.37 (m, 2H, CH₂—NH), 1.53 (p, J=6.9 Hz, 2H, CH₂—CH₂—NH), 1.38-1.11 (m, 30H alkyl chain), 0.88 (t, J=6.8 Hz, 3H, CH₃).

¹³C NMR (101 MHz, pyridine-d₅): δ174.6 (C-1), 106.3 (C-1′), 84.1 (C-4), 79.1 (C-5′), 78.8 (C-3′), 75.9 (C-2′), 73.8 (C-5), 73.3 (C-2), 73.2 (C-3), 72.1 (C-4′), 65.0 (C-6), 63.3 (C-6′), 39.8 (CH₂—NH), 32.5-23.3 (CH₂ alkyl chain), 14.6 (CH₃).

### N-Tetradecyl-D-Lactobionamide (Lac14)

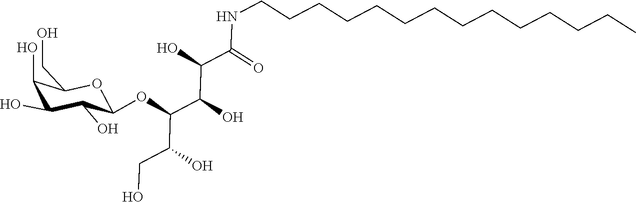

Yield: 76%
Appearance: solid white
Molecular formula: C₂₆H₅₁NO₁₁
MM: 553.68 g·mol⁻¹

¹H NMR (600 MHz, pyridine-d₅): δ8.18 (t, 1H, J=5.8 Hz, NH), 5.34 (t, 1H, J=3.0 Hz, H-3), 5.26 (d, 1H, J=2.4 Hz, H-2), 5.22 (d, 1H, J=7.7 Hz, H-1′), 4.81 (dd, 1H, J=4.1 Hz, J=6.9 Hz, H-4), 4.78-4.72 (m, 1H, H-5), 4.56-4.45 (m, 4H, H-2′, H-6′a, H-6a, H-6b), 4.43 (d, 1H, J=2.9 Hz, H-4′), 4.29 (dd, 1H, J=4.6 Hz, J=11.3 Hz, H-6′b), 4.14 (dd, 1H, J=3.2 Hz, J=9.4 Hz, H-3′), 4.08 (dd, 1H, J=5.4 Hz, J=6.4 Hz, H-5′), 3.52-3.38 (m, 2H, CH₂—NH), 1.58-1.47 (m, 2H, CH₂—CH₂—NH), 1.32-1.14 (m, 22H, CH₂ alkyl chain), 0.88 (t, 3H, J=6.8 Hz, CH₃)

¹³C NMR (151 MHz, pyridine-d₅): δ174.3 (C-1), 107.0 (C-1′), 84.8 (C-4), 77.9 (C-5′), 75.7 (C-3′), 74.2 (C-2), 73.8 (C-5), 73.5 (C-2′), 73.0 (C-3), 70.7 (C-4′), 64.9 (C-6), 63.0 (C-6′), 39.9 (CH₂—NH), 32.6-23.4 (CH₂ alkyl chain), 14.7 (CH₃).

### N-Hexadecyl-D-Lactobionamide (Lac16)

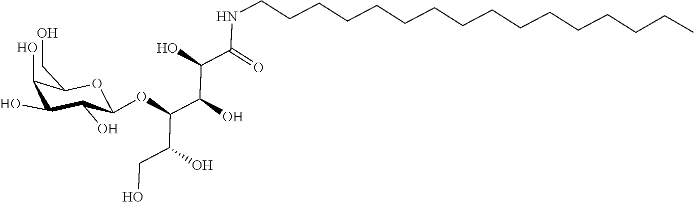

Yield: 69%
Appearance: solid white
Molecular formula: C₂₈H₅₅NO₁₁
MM: 581.74 g·mol⁻¹
Yield: 0.69 (EtOAc/MeOH/H₂O: 7/2/1)
[α]_{D}²⁰: +22.0° (MeOH, c=0.05)
ESI-HRMS: [M+H]⁺ 582.3852 (calculated: 582.3853 for C₂₈H₅₅NO₁₁)

¹H NMR (600 MHz, pyridine-d₅): δ8.19 (t, 1H, J=5.8 Hz, NH), 5.32 (t, 1H, J=3.0 Hz, H-3), 5.24 (d, 1H, J=2.4 Hz, H-2), 5.21 (d, 1H, J=7.7 Hz, H-1′), 4.80 (dd, 1H, J=4.1 Hz, J=6.9 Hz, H-4), 4.74-4.71 (m, 1H, H-5), 4.51-4.45 (m, 4H, H-2′, H-6′a, H-6a, H-6b), 4.42 (d, 1H, J=2.9 Hz, H-4′), 4.27 (dd, 1H, J=4.6 Hz, J=11.3 Hz, H-6′b), 4.12 (dd, 1H, J=3.2 Hz, J=9.4 Hz, H-3′), 4.06 (dd, 1H, J=5.4 Hz, J=6.4 Hz, H-5′), 3.48-3.38 (m, 2H, CH₂—NH), 1.55-1.48 (m, 2H, CH₂—CH₂—NH), 1.27-1.16 (m, 26H, CH₂ alkyl chain), 0.87 (t, 3H, J=6.8 Hz, CH₃)

¹³C NMR (151 MHz, pyridine-d₅): δ174.2 (C-1), 106.9 (C-1′), 84.6 (C-4), 77.8 (C-5′), 75.6 (C-3′), 74.1 (C-2), 73.7 (C-5), 73.4 (C-2′), 72.9 (C-3), 70.6 (C-4′), 64.8 (C-6), 62.9 (C-6′), 39.8 (CH₂—NH), 32.5-23.3 (CH₂ alkyl chain), 14.6 (CH₃).

### N-Octadecyl-D-Lactobionamide (Lac18)

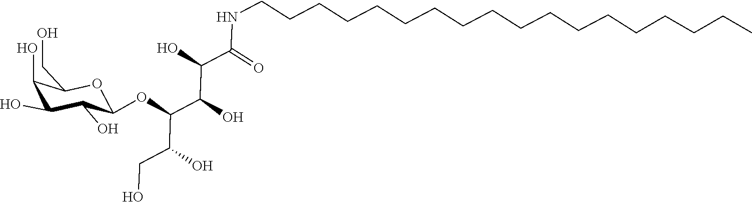

Yield: 86%
Appearance: solid white
Molecular formula: C₃₀H₅₉NO₁₁
MM: 609.80 g·mol⁻¹
Yield: 0.71 (EtOAc/MeOH/H₂O: 7/2/1)
[α]_{D}²⁰: +136.0° (MeOH, c=0.025)
ESI-HRMS: [M+H]⁺ 610.4166 (calculated: 610.4166 for C₃₀H₆₀NO₁₁)

¹H NMR (600 MHz, pyridine-d₅): δ8.19 (t, 1H, J=5.9 Hz, NH), 5.32 (br s, 1H, H-3), 5.24 (d, 1H, J=2.5 Hz, H-2), 5.21 (d, 1H, J=7.8 Hz, H-1′), 4.80 (dd, 1H, J=4.0 Hz, J=7.0 Hz, H-4), 4.74-4.72 (m, 1H, H-5), 4.52-4.45 (m, 4H, H-2′, H-6′a, H-6a, H-6b), 4.42 (d, 1H, J=3.1 Hz, H-4′), 4.27 (dd, 1H, J=4.4 Hz, J=11.2 Hz, H-6′b), 4.12 (dd, 1H, J=3.3 Hz, J=9.5 Hz, H-3′), 4.06 (dd, 1H, J=5.0 Hz, J=7.0 Hz, H-5′), 3.48-3.38 (m, 2H, CH₂—NH), 1.55-1.48 (m, 2H, CH₂—CH₂—NH), 1.29-1.16 (m, 30H, CH₂ alkyl chain), 0.87 (t, 3H, J=6.9 Hz, CH₃)

¹³C NMR (151 MHz, pyridine-d₅): δ174.2 (C-1), 106.9 (C-1′), 84.7 (C-4), 77.9 (C-5′), 75.6 (C-3′), 74.1 (C-2), 73.7 (C-5), 73.4 (C-2′), 72.9 (C-3), 70.6 (C-4′), 64.8 (C-6), 62.9 (C-6′), 39.8 (CH₂—NH), 32.5 (CH₂ alkyl chain), 30.6-23.3 (CH₂ alkyl chain), 14.6 (CH₃)

### N-Tetradecyl-Melibionamide (Mel14)

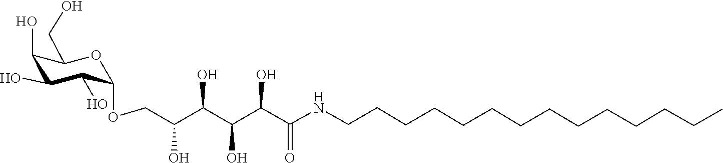

Yield: 60%
Appearance: solid white
Molecular formula: C₂₆H₅₁NO₁₁
MM: 553.35 g·mol⁻¹

¹H NMR (400 MHz, pyridine-d₅): δ8.39 (t, J=5.9 Hz, N—H), 5.46 (d, J=3.7 Hz, 1H, H-1′), 5.21 (t, J=3.2 Hz, 1H, H-3), 5.04 (d, J=3.3 Hz, 1H, H-2), 4.78-4.70 (m, 2H, H-4′, H-4), 4.68 (dd, J=9.9, 3.8 Hz, 1H, H-2′), 4.63-4.56 (m, 3H, H-6a,b, H-5′), 4.51 (dd, J=9.9, 3.1 Hz, 1H, H-3′), 4.42 (d, J=5.9 Hz, 2H, H-6′a,b), 4.38-4.32 (m, 1H, H-5), 3.56-3.37 (m, 2H, CH₂—N), 1.63-1.49 (m, 2H, CH₂—CH₂—N), 1.33-1.12 (m, 22H, CH₂ alkyl chain), 0.88 (t, J=6.7 Hz, 3H, CH₃).

¹³C NMR (101 MHz, pyridine-d₅): δ174.5 (C-1), 101.60 (C-1′), 75.3 (C-2), 75.1 (C-4), 73.2 (C-5′), 72.7 (C-3), 72.1 (C-3′), 71.4 (C-5, C-6), 71.4 (C-4′), 71.1 (C-2′), 63.0 (C-6′), 40.0 (CH₂—N), 33.0-23.4 (CH₂ alkyl chain), 14.7 (CH₃).

### N-Hexadecyl-Melibionamide (Mel16)

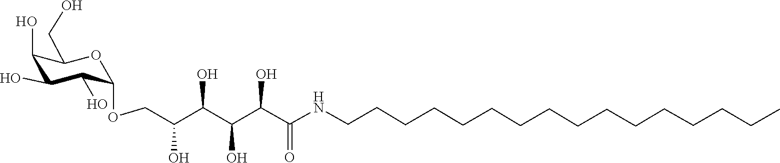

Yield: 44%
Appearance: solid white
Molecular formula: C₂₈H₅₅NO₁₁
MM: 581.38 g·mol⁻¹

¹H NMR (400 MHz, pyridine-d₅): δ8.39 (t, J=5.9 Hz, N—H), 5.45 (d, J=3.7 Hz, 1H, H-1′), 5.21 (t, J=3.2 Hz, 1H, H-3), 5.04 (d, J=3.3 Hz, 1H, H-2), 4.78-4.70 (m, 2H, H-4′, H-4), 4.67 (dd, J=9.9, 3.8 Hz, 1H, H-2′), 4.62-4.55 (m, 3H, H-6a,b, H-5′), 4.51 (dd, J=9.9, 3.1 Hz, 1H, H-3′), 4.41 (d, J=5.9 Hz, 2H, H-6′a,b), 4.38-4.30 (m, 1H, H-5), 3.54-3.40 (m, 2H, CH₂—N), 1.62-1.51 (m, 2H, CH₂—CH₂—N), 1.34-1.12 (m, 26H, CH₂ alkyl chain), 0.88 (t, J=6.7 Hz, 3H, CH₃).

¹³C NMR (101 MHz, pyridine-d₅): δ174.5 (C-1), 101.6 (C-1′), 75.3 (C-2), 75.1 (C-4), 73.2 (C-5′), 72.7 (C-3), 72.1 (C-3′), 71.4 (C-5, C-6), 71.4 (C-4′), 71.1 (C-2′), 63.0 (C-6′), 40.0 (CH₂—N), 33.0-23.4 (CH₂ alkyl chain), 14.8 (CH₃).

### N-Octadecyl-Melibionamide (Mel18)

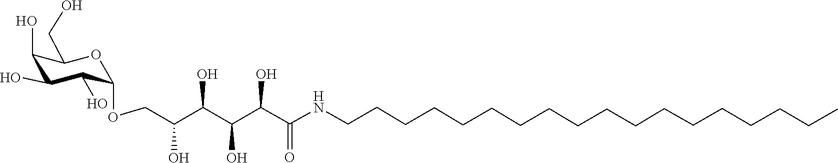

Yield: 38%
Appearance: solid white
Molecular formula: C₃₀H₅₉NO₁₁
MM: 609.41 g·mol⁻¹

¹H NMR (400 MHz, pyridine-d₅): δ8.39 (t, J=5.9 Hz, N—H), 5.46 (d, J=3.7 Hz, 1H, H-1′), 5.21 (t, J=3.2 Hz, 1H, H-3), 5.04 (d, J=3.3 Hz, 1H, H-2), 4.78-4.70 (m, 2H, H-4′, H-4), 4.68 (dd, J=9.9, 3.8 Hz, 1H, H-2′), 4.62-4.56 (m, 3H, H-6a,b, H-5′), 4.51 (dd, J=9.9, 3.1 Hz, 1H, H-3′), 4.42 (d, J=5.9 Hz, 2H, H-6′a,b), 4.38-4.31 (m, 1H, H-5), 3.54-3.40 (m, 2H, CH₂—N), 1.62-1.51 (m, 2H, CH₂—CH₂—N), 1.34-1.12 (m, 30H, CH₂ alkyl chain), 0.88 (t, J=6.7 Hz, 3H, CH₃).

¹³C NMR (101 MHz, pyridine-d₅): δ174.5 (C-1), 101.6 (C-1′), 75.3 (C-2), 75.1 (C-4), 73.2 (C-5′), 72.7 (C-3), 72.1 (C-3′), 71.4 (C-5, C-6), 71.4 (C-4′), 71.1 (C-2′), 63.0 (C-6′), 40.0 (CH₂—N), 33.0-23.4 (CH₂ alkyl chain), 14.8 (CH₃).

### II-2. c). General Procedure for Sulphation of N-Alkyl-Glyconamides

To 1 g of N-alkyl-glyconamide placed in a double-neck flask in 50 mL of anhydrous pyridine, are added 2×1.1 eq of 50% SO₃/pyridine complex active for glycobionamides and 3×1.1 eq of 50% SO₃/pyridine complex active for maltotrionamides. The progress of the reaction is monitored by reversed phase thin layer chromatography (C18 stationary phase, eluent methanol/water 6/4). After about an hour of reaction, 1.5 eq additional 50% SO₃/active pyridine is added to complete the reaction. The reaction is left under stirring overnight, then treated with a saturated sodium bicarbonate solution at pH≈9 before being evaporated to dryness. Disulphated compounds (N-alkyl-glyconamides) or trisulphated N-alkyl maltotrionamides are the majority products obtained in the reaction medium, with a minority formation of compounds that are not fully sulphated in the primary positions or contain an additional sulphate. The crude reaction is then purified by reverse-phase flash chromatography with a C18 80 g Reveleris column. The mobile phase is a water/methanol mixture with a flow rate of 40 mL/min and a gradient starting with 40% methanol and going up to 80% methanol in 10 min. The fractions of purified monosulphated, disulphated (very predominant) and trisulphated N-alkyl glycobionamide or trisulphated N-alkyl maltotrionamides are then evaporated to remove methanol and then freeze-dried.

### N-Tetradecyl-6,6′-Di-O-Sulfo-D-Maltobioamide Disodium (Mal14diS)

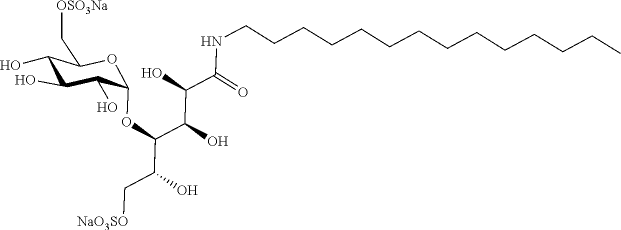

Yield: 61%
Appearance: solid white
Molecular formula: C₂₆H₄₉NNa₂O₁₇S₂
MM: 757.22 g·mol⁻¹
Yield: 0.47 (CH₃CN/H₂O: 3/7) TLC-C18
ESI-HRMS: [M+Na]⁺780.2133 (calculated: 780.2135 for C₂₆H₄₉NNa₃O₁₇S₂)

¹H NMR (600 MHz, D₂O): δ5.22 (d, J=3.9 Hz, 1H, H-1′), 4.41-4.10 (m, 8H, H-6′a, H-6′b, H-2, H-5, H-6a, H-6b, H-3, H-5′), 4.00 (dd, J=5.7, 3.8 Hz, 1H, H-4), 3.81 (t, J=9.5 Hz, 1H, H-3′), 3.65 (dd, J=9.9, 3.8 Hz, 1H, H-2′), 3.57 (t, J=9.6 Hz, 1H, H-4), 3.34-3.18 (m, 2H, CH₂—N), 1.57 (br s, 2H, CH₂—CH₂—N), 1.33 (br s, 22H, CH₂ alkyl chain), 0.92 (t, J=6.5 Hz, 3H, CH₃)

¹³C NMR (151 MHz, D₂O): δ173.4 (C-1), 100.5 (C-1′), 82.0 (C-4), 72.7 (C-3′), 71.8 (C-2), 71.7 (C-3), 71.5 (C-2′), 70.5 (C-5′), 69.8 (C-5), 68.9 (C-6), 68.8 (C-4′), 66.7 (C-6′), 39.4 (CH₂—N), 32.0-22.6 (CH₂ alkyl chain), 13.9 (CH₃).

### N-Hexadecyl-6,6′-Di-O-Sulfo-D-Maltobioamide Disodium (Mal16diS)

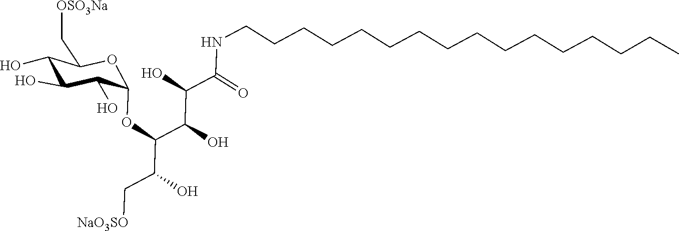

Yield: 55%
Appearance: solid white
Molecular formula: C₂₈H₅₃NNa₂O₁₇S₂
MM: 785.82 g·mol⁻¹
Yield: 0.52 (CH₃CN/H₂O: 6/4) TLC-C18
[α]_{D}²⁰: +53.6° (H₂O, c=0.5)
ESI-HRMS: [M+Na]⁺808.2463 (calculated: 808.2448 for C₂₃H₅₃NNa₃O₁₇S₂)

¹H NMR (400 MHz, D₂O): δ5.21 (d, J=3.9 Hz, 1H, H-1′), 4.40-4.09 (m, 8H, H-6′a, H-6′b, H-2, H-5, H-6a, H-6b, H-3, H-5′), 3.99 (dd, J=5.7, 3.8 Hz, 1H, H-4), 3.81 (t, J=9.5 Hz, 1H, H-3′), 3.64 (dd, J=9.9, 3.8 Hz, 1H, H-2′), 3.56 (t, J=9.6 Hz, 1H, H-4), 3.33-3.17 (m, 2H, CH₂—N), 1.56 (br s, 2H, CH₂—CH₂—N), 1.33 (br s, 26H, CH₂ alkyl chain), 0.92 (t, J=6.5 Hz, 3H, CH₃)

¹³C NMR (101 MHz, D₂O): δ173.4 (C-1), 100.5 (C-1′), 82.0 (C-4), 72.8 (C-3′), 71.8 (C-2), 71.7 (C-3), 71.5 (C-2′), 70.5 (C-5′), 69.8 (C-5), 68.9 (C-6), 68.8 (C-4′), 66.7 (C-6′), 39.4 (CH₂—N), 32.0-22.6 (CH₂ alkyl chain), 13.9 (CH₃).

### N-Hexadecyl-6 or 6′-O-Sulfo-D-Maltobioamide Sodium (Mal16monoS)

The monosulphated fraction of Mal16 is obtained from the crude Mal16diS purified on a flash column. This is a mixture of N-hexadecyl maltobionamide sulphated in position 6 and its homolog sulphated in position 6′

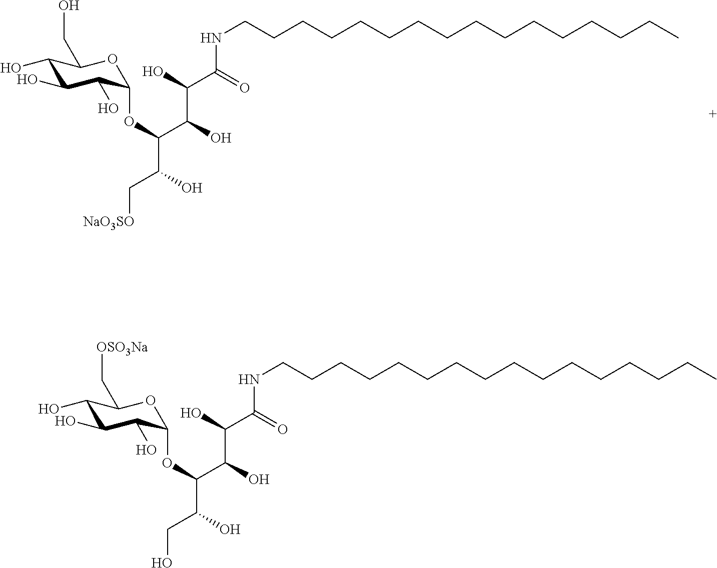

Yield: 40%
Appearance: solid white
Molecular formula: C₂₈H₅₄NNaO₁₄S
MM: 683.78 g·mol⁻¹

### N-Hexadecyl-Tri-O-Sulfo-D-Maltobioamide Trisodium (Mal16triS)

The trisulphated fraction of Mal16 is obtained from the crude Mal16diS purified on a flash column. It is a mixture of several N-hexadecyl maltobionamide compounds disulphated in position 6 and 6′ comprising a third sulphate on one of the secondary positions.

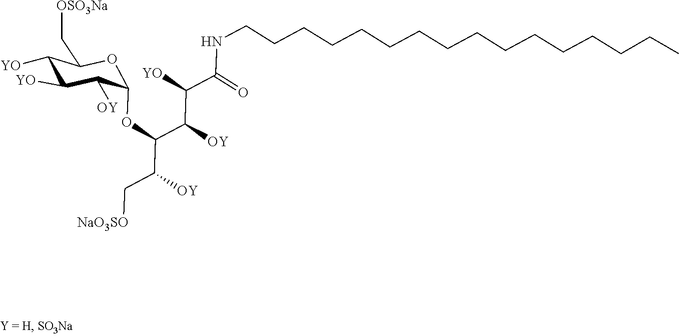

Yield: 6%
Appearance: solid white
Molecular formula: C₂₈H₅₂NNa₃O₂₀S₃
MM: 887.86 g·mol⁻¹

### N-Octadecyl-6,6′-Di-O-Sulfo-D-Maltobionamide Disodium (Mal18diS)

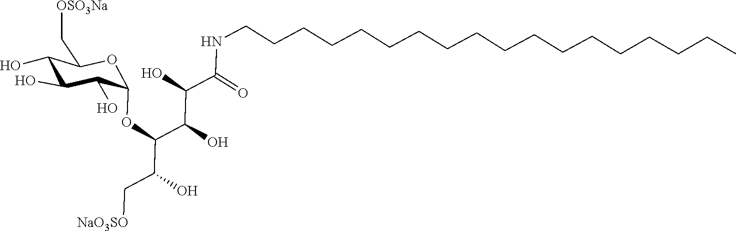

Yield: 49%
Appearance: solid white
Molecular formula: C₃₀H₅₇NNa₂O₁₇S₂
MM: 813.88 g·mol⁻¹
Yield: 0.44 (CH₃CN/H₂O: 6/4) TLC-C18
[α]_{D}²⁰: +39.2° (H₂O, c=0.125)
ESI-HRMS: [M+Na]⁺836.2776 (calculated: 836.2761 for C₃₀H₅₇NNa₃O₁₇S₂)

¹H NMR (400 MHz, D₂O): δ5.23 (d, J=3.8 Hz, 1H, H-1′), 4.45-4.08 (m, 8H, H-6′a, H-6′b, H-2, H-5, H-6a, H-6b, H-3, H-5′), 4.01 (t, J=4.8 Hz, 1H, H-4), 3.83 (t, J=9.5 Hz, 1H, H-3′), 3.67 (dd, J=9.9, 3.5 Hz, 1H, H-2′), 3.58 (t, J=9.5 Hz, 1H, H-4′), 3.41-3.10 (m, 2H, CH₂—N), 1.58 (br s, 2H, CH₂—CH₂—N), 1.33 (br s, 30H, CH₂ alkyl chain), 0.93 (t, J=6.5 Hz, 3H, CH₃).

¹³C NMR (D₂O, 101 MHz): δ173.4 (C-1), 100.5 (C-1′), 82.0 (C-4), 72.8 (C-3′), 71.8 (C-2), 71.6 (C-3), 71.5 (C-2′), 70.5 (C-5′), 69.8 (C-5), 68.9 (C-6), 68.9 (C-4′), 66.7 (C-6′), 39.5 (CH₂—N), 32.1-22.7 (CH₂ alkyl chain), 13.9 (CH₃).

### N-Octadecyl-6 or 6′-O-Sulfo-D-Maltobioamide Sodium (Mal18monoS)

The monosulphated fraction of Mal18 is obtained from the crude Mal18diS purified on a flash column. This is a mixture of N-octadecyl maltobionamide sulphated at position 6 and its homolog sulphated at position 6′

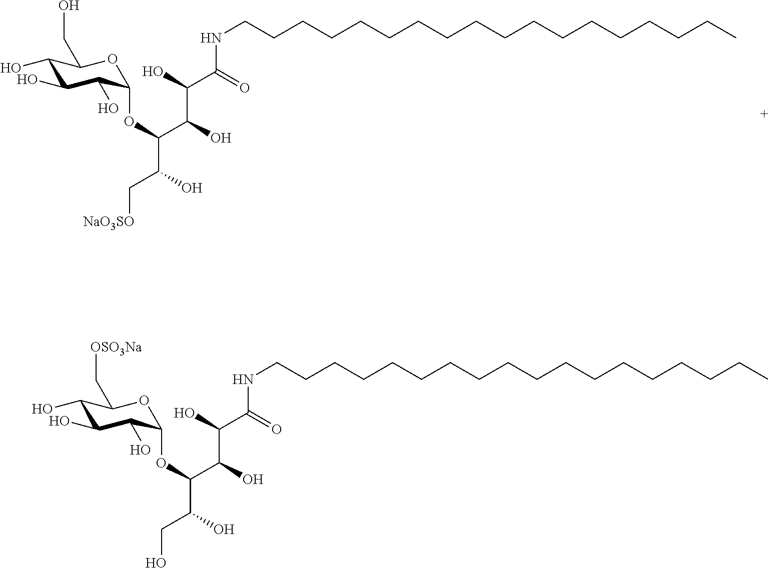

Yield: 37%
Appearance: solid white
Molecular formula: C₃₀H₅₈NNaO₁₄S
MM: 711.84 g·mol⁻¹

### N-Octadecyl-Tri-O-Sulfo-D-Maltobioamide Trisodium (Mal18triS)

The trisulphated fraction of Mal18 is obtained from the crude Mal18diS purified on a flash column. It is a mixture of several N-octadecyl maltobionamide compounds disulphated in position 6 and 6′ comprising a third sulphate on one of the secondary positions.

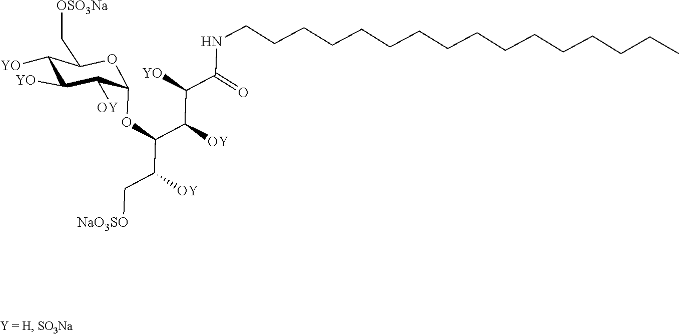

Yield: 5%
Appearance: solid white
Molecular formula: C₃₀H₅₆NNa₃O₂₀S₃
MM: 915.91 g·mol⁻¹
   N-Nonadecyl-6,6′-di-O-sulfo-D-maltobionamide disodium (Mal19diS)

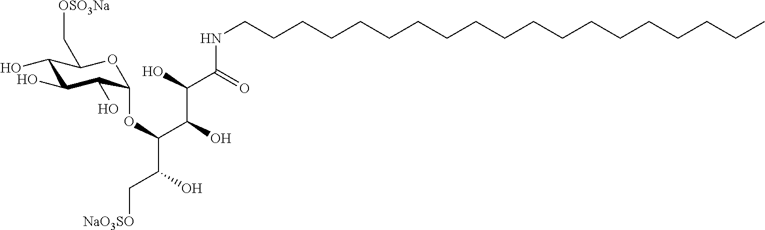

Yield: 47%
Appearance: solid white
Molecular formula: C₃₁1H₅₉NNa₂O₁₇S₂
MM: 827.90 g·mol⁻¹

¹H NMR (400 MHz, D₂O): δ5.22 (d, J=3.8 Hz, 1H, H-1′), 4.42-4.09 (m, 8H, H-6′a, H-6′b, H-2, H-5, H-6a, H-6b, H-3, H-5′), 4.00 (t, J=4.8 Hz, 1H, H-4), 3.82 (t, J=9.5 Hz, 1H, H-3′), 3.65 (dd, J=9.9, 3.5 Hz, 1H, H-2′), 3.57 (t, J=9.5 Hz, 1H, H-4′), 3.35-3.15 (m, 2H, CH₂—N), 1.57 (br s, 2H, CH₂—CH₂—N), 1.33 (br s, 32H, CH₂ alkyl chain), 0.91 (t, J=6.5 Hz, 3H, CH₃).

¹³C NMR (D₂O, 101 MHz): δ173.4 (C-1), 100.5 (C-1′), 82.0 (C-4), 72.7 (C-3′), 71.9 (C-2), 71.5 (C-3), 71.5 (C-2′), 70.5 (C-5′), 69.8 (C-5), 68.9 (C-6), 68.9 (C-4′), 66.7 (C-6′), 39.5 (CH₂—N), 32.1-22.7 (CH₂ alkyl chain), 13.9 (CH₃).

### N-Eicosanyl-6,6′-Di-O-Sulfo-D-Maltobionamide Disodium (Mal20diS)

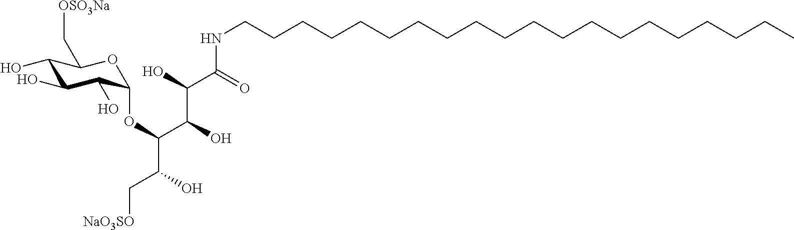

Yield: 32%
Appearance: solid white
Molecular formula: C₃₂H₆₁NNa₂O₁₇S₂
MM: 841.32 g·mol⁻¹

¹H NMR (400 MHz, D₂O): δ5.23 (d, J=3.8 Hz, 1H, H-1′), 4.42-4.08 (m, 8H, H-6′a, H-6′b, H-2, H-5, H-6a, H-6b, H-3, H-5′), 4.01 (t, J=4.8 Hz, 1H, H-4), 3.82 (t, J=9.5 Hz, 1H, H-3′), 3.66 (dd, J=9.9, 3.5 Hz, 1H, H-2′), 3.57 (t, J=9.5 Hz, 1H, H-4′), 3.36-3.14 (m, 2H, CH₂—N), 1.57 (br s, 2H, CH₂—CH₂—N), 1.33 (br s, 34H, CH₂ alkyl chain), 0.91 (t, J=6.5 Hz, 3H, CH₃).

¹³C NMR (D₂O, 101 MHz): δ173.4 (C-1), 100.5 (C-1′), 82.0 (C-4), 72.8 (C-3′), 71.8 (C-2), 71.6 (C-3), 71.5 (C-2′), 70.5 (C-5′), 69.8 (C-5), 68.9 (C-6), 68.9 (C-4′), 66.7 (C-6′), 39.5 (CH₂—N), 32.1-22.7 (CH₂ alkyl chain), 13.9 (CH₃).

### N-Hexadecyl-Per-O-Sulfo-D-Maltobioamide Sodium Salt (Mal16perS)

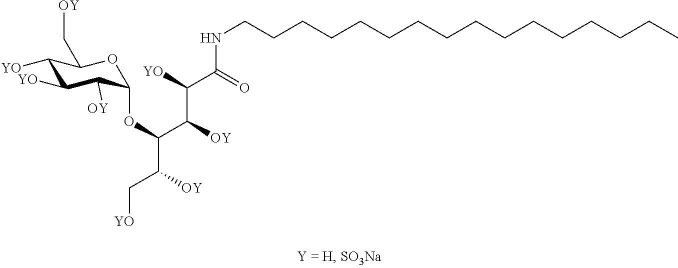

The N-hexadecyl-D-maltobionamide (Mal16) compound (200 mg, 0.34 mmol) and 20 equivalents of 97% SO₃/active pyridine are introduced into a 20 mL zirconia bowl containing 80 zirconia balls with a diameter of 5 mm, then into the P7PL Fritsch planetary mill. The mixture is ground at 400 rpm for 48 cycles of 5 min. Then 30 equivalents of NaHCO₃ (578 mg, 6.88 mmol) are introduced into the bowl and the mixture is ground again for 6 cycles of 5 minutes. The crude reaction is then dissolved in milliQ water and dialyzed using a 1000 kD cutoff membrane (3×500 mL). After freeze-drying, a white solid is obtained and analysed by high-resolution mass spectrometry. Mal16perS is composed of a mixture of tri, tetra, penta, hexa, hepta and octa sulphated N-hexadecyl-D-maltobionamides (524 mg).

### N-Tetradecyl-6,6′-Di-O-Sulfo-D-Cellobioamide Disodium (Cell14diS)

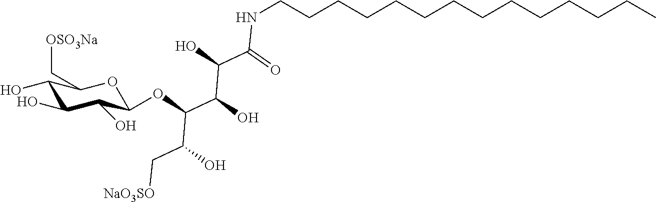

Yield: 69%
Appearance: solid white
Molecular formula: C₂₆H₄₉NNa₂O₁₇S₂
MM: 757.22 g·mol⁻¹

¹H NMR (400 MHz, D₂O): δ4.69 (d, J=7.9 Hz, 1H, H-1′), 4.44 (bs, 1H, H-2), 4.40-4.33 (m, 4H, H-6′a, H-6a, H-6b, H-3), 4.33-4.10 (m, 2H, H-6′b, H-5), 4.06 (dd, J=7.7, 3.2 Hz, 1H, H-4′), 3.79-3.71 (m, 1H, H-5), 3.60-3.48 (m, 2H, H-3′, H-4′), 3.40 (t, J=8.4 Hz, 1H, H-2′), 3.32-3.20 (m, 2H, CH₂—N), 1.61-1.51 (m, 2H, CH₂—CH₂—N), 1.33 (s, 22H, CH₂ alkyl chain), 0.91 (t, J=6.4 Hz, 3H, CH₃).

¹³C NMR (101 MHz, D₂O): δ173.5 (C-1), 102.6 (C-1′), 80.5 (C-4), 75.3 (C-3′), 73.7 (C-5′), 73.2 (C-2′), 72.9 (C-2), 69.5 (C-3), 69.3 (C-4′), 68.9 (C-5, C-6), 66.8 (C-6′), 39.3 (CH₂—N), 31.7-22.4 (CH₂ alkyl chain), 13.8 (CH₃).

### N-Hexadecyl-6,6′-Di-O-Sulfo-D-Cellobioamide Disodium (Cell6diS)

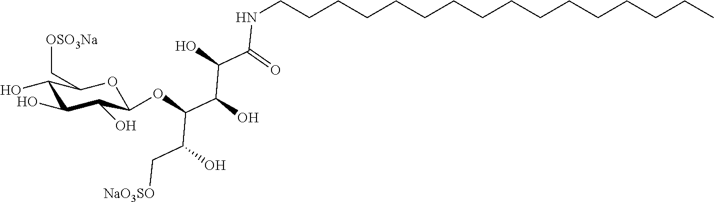

Yield: 59%
Appearance: solid white
Molecular formula: C₂₈H₅₃NNa₂O₁₇S₂
MM: 785.82 g·mol⁻¹
Yield: 0.6 (H₂O/CH₃CN: 65/35) TLC-C18
ESI-HRMS: [M+Na]⁺808.2472 (calculated: 808.2448 for C₂₃H₅₃NNa₃O₁₇S₂)

¹H NMR (400 MHz, D₂O): δ4.69 (d, J=7.9 Hz, 1H, H-1′), 4.45 (bs, 1H, H-2), 4.40-4.27 (m, 4H, H-6′a, H-6a, H-6b, H-3), 4.24-4.13 (m, 2H, H-6′b, H-5), 4.07 (dd, J=7.7, 3.2 Hz, 1H, H-4′), 3.77-3.73 (m, 1H, H-5), 3.58-3.48 (m, 2H, H-3′, H-4′), 3.44 (t, J=8.4 Hz, 1H, H-2′), 3.32-3.18 (m, 2H, CH₂—N), 1.58-1.54 (m, 2H, CH₂—CH₂—N), 1.33 (s, 26H, CH₂ alkyl chain), 0.93 (t, J=6.4 Hz, 3H, CH₃).

¹³C NMR (D₂O, 101 MHz): δ173.5 (C-1), 102.6 (C-1′), 80.5 (C-4), 75.3 (C-3′), 73.9 (C-5′), 73.2 (C-2′), 72.9 (C-2), 69.7 (C-3), 69.5 (C-4′), 68.9 (C-5, C-6), 67.0 (C-6′), 39.4 (CH₂—N), 32.0-22.7 (CH₂ alkyl chain), 14.0 (CH₃).

### N-Octadecyl-6,6′-Di-O-Sulfo-D-Cellobioamide Disodium (Cell18diS)

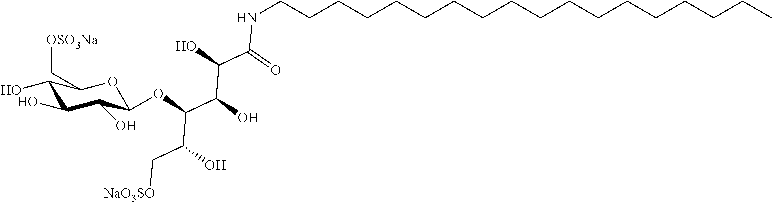

Yield: 66%
Appearance: solid white
Molecular formula: C₃₀H₅₇NNa₂O₁₇S₂
MM: 813.88 g·mol⁻¹
Yield: 0.22 (CH₃CN/H₂O: 2/8) TLC-C18
ESI-HRMS: [M+Na]⁺836.2803 (calculated: 836.2761 for C₃₀H₅₇NNa₃O₁₇S₂)

¹H NMR (400 MHz, D₂O): δ4.69 (d, J=7.9 Hz, 1H, H-1′), 4.45 (bs, 1H, H-2′), 4.41-4.26 (m, 4H, H-6′a, H-6a, H-6b, H-3), 4.24-4.13 (m, 2H, H-6′b, H-5), 4.07 (dd, J=7.7, 3.2 Hz, 1H, H-4′), 3.78-3.72 (m, 1H, H-5), 3.60-3.48 (m, 2H, H-3′, H-4′), 3.41 (t, J=8.4 Hz, 1H, H-2′), 3.32-3.18 (m, 2H, CH₂—N), 1.63-1.52 (m, 2H, CH₂—CH₂—N), 1.34 (s, 30H, CH₂ alkyl chain), 0.93 (t, J=6.4 Hz, 3H, CH₃).

¹³C NMR (101 MHz, D₂O): δ173.4 (C-1), 102.6 (C-1′), 80.5 (C-4), 75.3 (C-3′), 73.9 (C-5′), 73.2 (C-2′), 72.8 (C-2), 69.7 (C-3), 69.5 (C-4′), 68.9 (C-5, C-6), 67.0 (C-6′), 39.4 (CH₂—N), 32.0-22.7 (CH₂ alkyl chain), 13.9 (CH₃).

### N-Tetradecyl-6,6′-Di-O-Sulfo-Lactobioamide Disodium (Lac14diS)

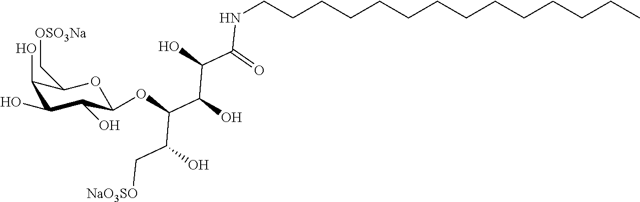

Yield: 72%
Appearance: solid white
Molecular formula: C₂₆H₄₉NNa₂O₁₇S₂
MM: 757.22 g·mol⁻¹
Yield: 0.23 (H₂O/CH₃CN: 7/3) TLC-C18
ESI-HRMS: [M+Na]⁺780.2146 (calculated: 780.2135 for C₂₆H₄₉NNa₃O₁₇S₂)

¹H NMR (400 MHz, D₂O): δ4.63 (d, J=7.7 Hz, 1H, H-1′), 4.45 (bs, 1H, H-2), 4.42-4.26 (m, 3H, H-6a, H-6b, H-3), 4.24 (d, J=5.9 Hz, 2H, H-6′a, H-6′b), 4.20-4.13 (m, 1H, H-5), 4.08 (dd, J=8.0, 2.9 Hz, 1H, H-4), 4.09-3.94 (m, 2H, H-4′, H-5), 3.72 (dd, J=10.0, 3.3 Hz, 1H, H-3′), 3.62 (dd, J=9.9, 7.6 Hz, 1H, H-2′), 3.36-3.16 (m, 2H, CH₂—N), 1.63-1.50 (m, 2H, CH₂—CH₂—N), 1.32 (s, 22H, CH₂ alkyl chain), 0.92 (t, J=6.4 Hz, 3H, CH₃).

¹³C NMR (101 MHz, D₂O): δ173.5 (C-1), 103.1 (C-1′), 80.4 (C-4), 72.9 (C-2), 72.8 (C-4′), 72.4 (C-3′), 70.9 (C-2′), 69.7 (C-3), 69.1 (C-5, C-6), 68.3 (C-5′), 66.7 (C-6′), 39.4 (CH₂—N), 32.0-22.6 (CH₂ alkyl chain), 13.9 (CH₃).

### N-Hexadecyl-6,6′-Di-O-Sulfo-Lactobioamide Disodium (Lac16diS)

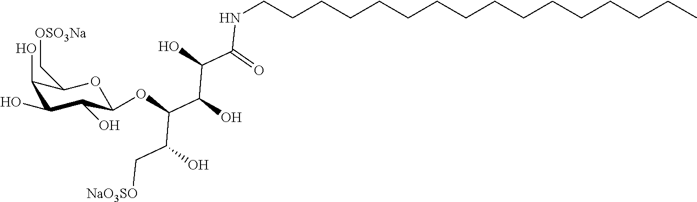

Yield: 72%
Appearance: solid white
Molecular formula: C₂₈H₅₃NNa₂O₁₇S₂
MM: 785.82 g·mol⁻¹
Yield: 0.23 (H₂O/CH₃CN: 7/3) TLC-C18
ESI-HRMS: [M+Na]⁺808.2473 (calculated: 808.2448 for C₂₃H₅₃NNa₃O₁₇S₂)

¹H NMR (400 MHz, D₂O): δ4.64 (d, J=7.7 Hz, 1H, H-1′), 4.46 (bs, 1H, H-2), 4.43-4.28 (m, 3H, H-6a, H-6b, H-3), 4.24 (d, J=5.9 Hz, 2H, H-6′a, H-6′b), 4.20-4.13 (m, 1H, H-5), 4.08 (dd, J=8.0, 2.9 Hz, 1H, H-4), 4.05-3.99 (m, 2H, H-4′, H-5), 3.72 (dd, J=10.0, 3.3 Hz, 1H, H-3′), 3.63 (dd, J=9.9, 7.6 Hz, 1H, H-2′), 3.36-3.16 (m, 2H, CH₂—N), 1.64-1.52 (m, 2H, CH₂—CH₂—N), 1.33 (s, 26H, CH₂ alkyl chain), 0.92 (t, J=6.4 Hz, 3H, CH₃).

¹³C NMR (101 MHz, D₂O): δ173.5 (C-1), 103.1 (C-1′), 80.4 (C-4), 72.9 (C-2), 72.8 (C-4′), 72.4 (C-3′), 70.9 (C-2′), 69.7 (C-3), 69.0 (C-5, C-6), 68.3 (C-5′), 66.7 (C-6′), 39.4 (CH₂—N), 32.0-22.7 (CH₂ alkyl chain), 13.9 (CH₃).

### N-Octadecyl-6,6′-Di-O-Sulfo-Lactobioamide Disodium (Lac18diS)

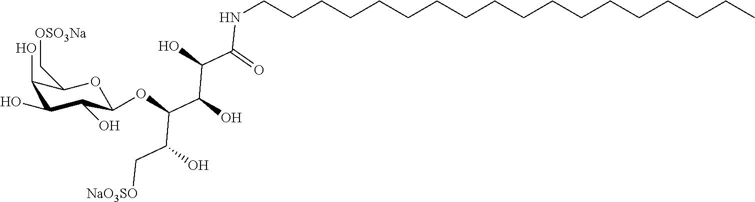

Yield: 64%
Appearance: solid white
Molecular formula: C₃₀H₅₇NNa₂O₁₇S₂
MM: 813.88 g·mol⁻¹
Yield: 0.36 (H₂O/CH₃CN: 7/3) TLC-C18
ESI-HRMS: [M+Na]⁺836.2798 (calculated: 836.2761 for C₃₀H₅₇NNa₃O₁₇S₂)

¹H NMR (D₂O, 400 MHz): δ 4.65 (d, J=7.6 Hz, 1H, H-1′), 4.47 (bs, 1H, H-2), 4.43-4.28 (m, 3H H-6a, H-6b, H-3), 4.24 (d, J=5.7 Hz, 2H, H-6′a, H-6′b), 4.18 (d, J=7.1 Hz, 1H, H-5), 4.15-4.06 (m, 1H, H-4), 4.07-4.00 (m, 2H, H-4′, H-5), 3.73 (dd, J=10.1, 3.0 Hz, 1H, H-3′), 3.65 (dd, J=9.8, 7.6 Hz, 1H, H-2′), 3.36-3.16 (m, 2H, CH₂—N), 1.65-1.49 (m, 2H, CH₂—CH₂—N), 1.34 (s, 30H, CH₂ alkyl chain), 0.93 (t, J=6.4 Hz, 3H, CH₃).

¹³C NMR (D₂O, 101 MHz): δ173.5 (C-1), 103.1 (C-1′), 80.3 (C-4), 72.9 (C-2), 72.8 (C-4′), 72.4 (C-3′), 70.9 (C-2′), 69.7 (C-3), 68.9 (C-5, C-6), 68.3 (C-5′), 66.7 (C-6′), 39.5 (CH₂—N), 32.0-22.7 (CH₂ alkyl chain), 13.9 (CH₃).

### N-Tetradecyl-6′-O-Sulfo-Melibioamide Sodium (Mel14monoS)

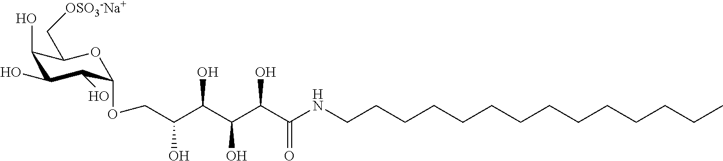

Yield: 48%
Appearance: solid white
Molecular formula: C₂₆H₅₀NNaO₁₄S
MM: 655.28 g·mol⁻¹
Yield: 0.32 (H₂O/CH₃CN: 7/3) TLC-C18
ESI-HRMS: [M+Na]⁺678.2750 (calculated: 678.2747 for C₂₆H₅₀NNa₃O₁₄S)

¹H NMR (400 MHz, D₂O): δ5.03 (d, J=3.7 Hz, 1H, H-1′), 4.35 (bs, 1H, H-2), 4.30-4.14 (m, 4H, H-6′a, H-6′b, H-5′, H-3), 4.08 (d, J=3.2 Hz, 1H, H-4′), 4.01-3.90 (m, 4H, H-3′, H-5, H-4, H-2′), 3.75 (dd, J=22, 7.2 Hz, 1H, H-6a, H-6b), 3.38-3.12 (m, 2H, CH₂—N), 1.66-1.50 (m, 2H, CH₂—CH₂—N), 1.47-1.21 (s, 22H, CH₂ alkyl chain), 0.92 (t, J=6.4 Hz, 3H, CH₃).

¹³C NMR (101 MHz, D₂O): δ173.7 (C-1), 98.4 (C-1′), 73.6 (C-2), 72.3 (C-4), 70.1 (C-3), 69.6 (C-5), 69.3 (C-3′), 69.2 (C-4′), 68.6 (C-6, C-5′), 68.4 (C-2′), 67.4 (C-6′), 39.4 (CH₂—N), 32.1-22.7 (CH₂ alkyl chain), 13.9 (CH₃).

### N-Hexadecyl-6′-O-Sulfo-Melibioamide Sodium (Mel16monoS)

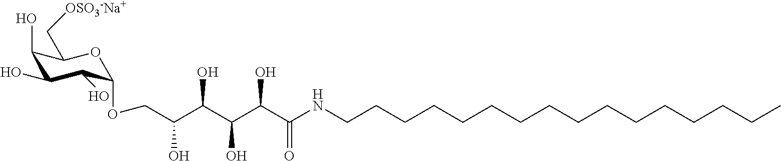

Yield: 53%
Appearance: solid white
Molecular formula: C₂₈H₅₄NNaO₁₄S
MM: 683.32 g·mol⁻¹
Yield: 0.22 (H₂O/CH₃CN: 7/3) TLC-C18
ESI-HRMS: [M+Na]⁺706.3082 (calculated: 706.3060 for C₂₈H₅₄NNa₃O₁₄S)

¹H NMR (400 MHz, D₂O): δ5.03 (d, J=3.7 Hz, 1H, H-1′), 4.35 (bs, 1H, H-2), 4.29-4.13 (m, 4H, H-6′a, H-6′b, H-5′, H-3), 4.08 (d, J=3.2 Hz, 1H, H-4′), 4.01-3.90 (m, 4H, H-3′, H-5, H-4, H-2′), 3.75 (dd, J=22, 7.2 Hz, 1H, H-6a, H-6b), 3.37-3.14 (m, 2H, CH₂—N), 1.63-1.52 (m, 2H, CH₂—CH₂—N), 1.46-1.18 (s, 26H, CH₂ alkyl chain), 0.91 (t, J=6.4 Hz, 3H, CH₃).

¹³C NMR (101 MHz, D₂O): δ173.7 (C-1), 98.4 (C-1′), 73.6 (C-2), 72.3 (C-4), 70.1 (C-3), 69.6 (C-5), 69.3 (C-3′), 69.1 (C-4′), 68.7 (C-6), 68.6 (C-5′), 68.4 (C-2′), 67.4 (C-6′), 39.4 (CH₂—N), 32.1-22.7 (CH₂ alkyl chain), 13.9 (CH₃).

### N-Octadecyl-6′-O-Sulfo-Melibioamide Sodium (Mel18monoS)

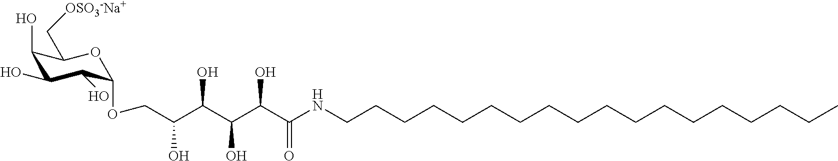

Yield: 38%
Appearance: solid white
Molecular formula: C₃₀H₅₈NNaO₁₄S
MM: 711.35 g·mol⁻¹
Yield: 0.16 (H₂O/CH₃CN: 7/3) TLC-C18
ESI-HRMS: [M]⁻ 688.3569 (calculated: 688.3578 for C₃₀H₅₈NO₁₄S)

¹H NMR (400 MHz, D₂O): δ5.03 (d, J=3.7 Hz, 1H, H-1′), 4.35 (bs, 1H, H-2), 4.28-4.14 (m, 4H, H-6′a, H-6′b, H-5′, H-3), 4.08 (d, J=3.2 Hz, 1H, H-4′), 4.01-3.81 (m, 4H, H-3′, H-5, H-4, H-2′), 3.75 (dd, J=22, 7.2 Hz, 1H, H-6a, H-6b), 3.37-3.14 (m, 2H, CH₂—N), 1.63-1.52 (m, 2H, CH₂—CH₂—N), 1.46-1.18 (s, 30H, CH₂ alkyl chain), 0.91 (t, J=6.4 Hz, 3H, CH₃).

¹³C NMR (101 MHz, D₂O): δ173.7 (C-1), 98.5 (C-1′), 73.7 (C-2), 72.3 (C-4), 70.1 (C-3), 69.6 (C-5), 69.3 (C-3′), 69.1 (C-4′), 68.7 (C-6), 68.6 (C-5′), 68.4 (C-2′), 67.4 (C-6′), 39.4 (CH₂—N), 32.1-22.7 (CH₂ alkyl chain), 13.9 (CH₃).

### II-3. Synthesis of Oxidized Glycobionamides

The synthesis of oxidized glycobionamides was carried out according to the reaction diagram below (Diagram 2).

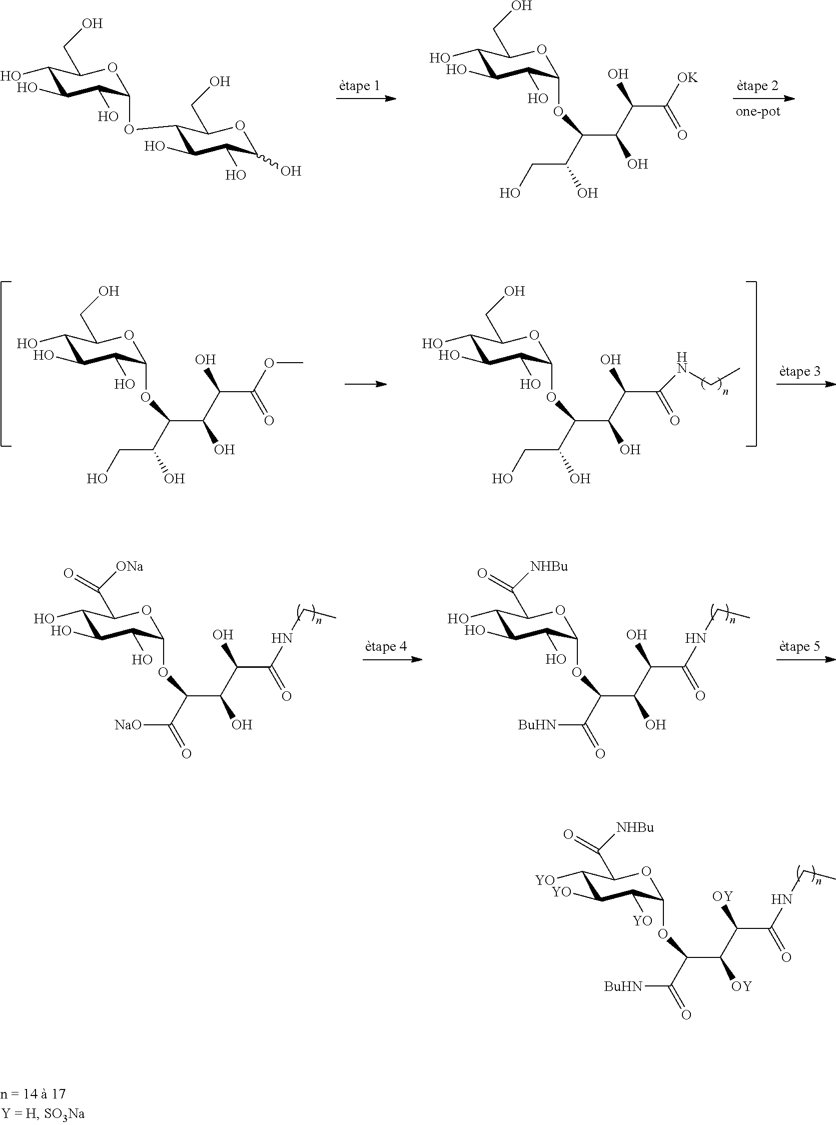

### Diagram 2: Synthetic Pathway of Oxidized Glycobionamides

The synthesis of oxidized glycobionamides involves three steps, the first two of which are identical to the synthesis of sulphated derivatives: oxidation of the anomeric position and one-pot esterification-aminolysis to graft the lipid chain via an amide bond.

Step 3: Oxidation of the primary hydroxyls of the disaccharide by the TEMPO/BAIB and NaHCO₃ system in a CH₃CN/H₂O mixture (48 h) (Lu, H. et al., Molecules 2016, 21(10), 1301/1-1301/15). The NMR and HRMS analysis shows that a loss of carbon —C6 (by decarboxylation) has occurred, similar to what was described in the literature, with a pyruvate motif (Coggins, A. J. et al., Nature Chemistry 2017, 9(4), 310-317; Lee Y et al., Chem Commun 2014, 50).

For the Mal16diBu-perS compound, two additional steps are necessary: esterification-aminolysis (step 4) followed by persulphation (step 5) (Bu=butyl in diagram 2).

### N-Hexadecyl-D-Glucuronyl-(α-1,4)-D-Xyluronamide (Mal16diOx)

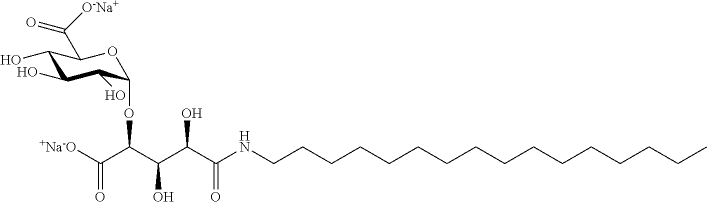

The N-hexadecyl-maltobionamide derivative (0.60 mmol, 350 mg) is dissolved in a mixture of acetonitrile (4 mL) and mQ H₂O (4 mL). The mixture is stirred at 0° C. and TEMPO (1.20 mmol, 187 mg) is added, followed by BAIB (6.02 mmol, 1.94 g) and NaHCO₃ (3.01 mmol, 253 mg). Then, the mixture is stirred at room temperature for 72 h. Ethanol (10 mL) is added and the reaction medium is diluted with EtOAc (100 mL) and mQ H₂O (100 mL). The aqueous phase is isolated and extracted with EtOAc (2×100 mL), then freeze-dried. The crude reaction is then purified by reversed phase chromatography. The elution gradient is H₂O/MeCN: 100/0 to 0/100 in 20 min. After evaporation and freeze drying, the Mal16diOx compound is obtained in the form of a white powder, with a yield of 8% (31 mg).
M: 623.65 g·mol⁻¹
Formula: C₂₇H₄₇NNa₂O₁₂
Yield: 0.51 (H₂O/MeCN: 5/5)
HRMS: 602.3150 (found 602.3152 for C₂₇H₄₉NO₁₂Na)

¹H NMR (D₂O, 400 MHz): 5.14 (m, 1H, H_{Glc1′}), 4.20 (m, 3H, H_{Glc2}, H_{Glc3} and H_{Glc5′}), 4.10 (d, 1H, J=6.0 Hz, H_{Glc4}), 3.93 (t, 1H, J=9.4 Hz, H_{Glc3′}), 3.65 (d, 1H, J=9.6 Hz, H_{Glc2′}), 3.50 (t, 1H, J=9.5 Hz, H_{Glc4′}), 3.22 (m, 2H, CH₂—NH), 1.55 (m, 2H, CH₂—CH₂—NH), 1.32 (m, 26H, CH₂ alkyl chain), 0.90 (t, 3H, J=6.2 Hz, CH₃ alkyl chain)

¹³C NMR (D₂O, 100 MHz): 177.22 (C_{Glc5}), 176.93 (C_{Glc6′}), 173.68 (C_{Glc1}), 100.66 (C_{Glc1′}), 83.33 (C_{Glc4}), 72.84 (C_{Glc2} or C_{Glc3}), 72.55 (C_{Glc2} or C_{Glc3}), 72.44 (C_{Glc3′}), 72.07 (C_{Glc4′}), 71.59 (C_{Glc5′}), 71.29 (C_{Glc2′}), 39.38 (CH₂—NH), 32.09 (CH₂ alkyl chain), 30.16-29.11 (CH₂ alkyl chain), 27.14 (CH₂ alkyl chain), 22.71 (CH₂ alkyl chain), 13.86 (CH₃ alkyl chain).

### N-Octadecyl-D-Glucuronyl-(α-1,4)-D-Xyluronamide (Mal18diOx)

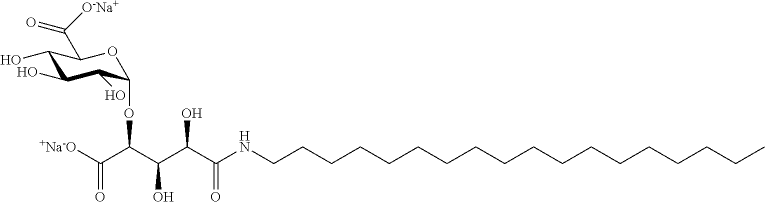

The N-hexadecyl-maltobionamide derivative (0.82 mmol, 500 mg) is dissolved in a mixture of acetonitrile (5 mL) and mQ H₂O (5 mL). The mixture is stirred at 0° C. and TEMPO (1.64 mmol, 256 mg) is added, followed by BAIB (8.20 mmol, 2.64 g) and NaHCO₃ (4.10 mmol, 344 mg). Then, the mixture is stirred at room temperature for 48 h. Ethanol (5 mL) is added and the reaction medium is diluted with EtOAc (200 mL) and mQ H₂O (200 mL). The aqueous phase is isolated and extracted with EtOAc (2×200 mL), then freeze-dried. The crude reaction is then purified by reversed phase chromatography. The elution gradient is H₂O/MeCN: 100/0 to 0/100 in 20 min. After evaporation and freeze drying, the Mal18diOx compound is obtained in the form of a white powder, with a yield of 17% (90 mg).
M: 651.70 g·mol⁻¹
Formula: C₂₉H₅₁NNa₂O₁₂
Yield: 0.48 (H₂O/MeCN: 5/5)
HRMS: 630.3466 (found 630.3465 for C₂₉H₅₃NO₁₂Na)

¹H NMR (D₂O, 400 MHz): 5.14 (m, 1H, H_{Glc1′}), 4.20 (m, 3H, H_{Glc2}, H_{Glc3} and H_{Glc5′}), 4.10 (d, 1H, J=6.0 Hz, H_{Glc4}), 3.94 (t, 1H, J=8.7 Hz, H_{Glc3′}), 3.66 (d, 1H, J=9.6 Hz, H_{Glc2′}), 3.50 (t, 1H, J=9.5 Hz, H_{Glc4′}), 3.21 (m, 2H, CH₂—NH), 1.54 (m, 2H, CH₂—CH₂—NH), 1.32 (m, 30H, CH₂ alkyl chain), 0.88 (t, 3H, J=6.2 Hz, CH₃ alkyl chain)

¹³C NMR (D₂O, 100 MHz): 177.31 (C_{Glc5}), 176.94 (C_{Glc6′}), 173.68 (C_{Glc1}), 100.71 (C_{Glc1′}), 82.38 (C_{Glc4}), 72.80 (C_{Glc2} or C_{Glc3}), 72.55 (C_{Glc2} or C_{Glc3} or C_{Glc3′}), 72.04 (C_{Glc4′}), 71.74 (C_{Glc5′}), 71.23 (C_{Glc2′}), 39.43 (CH₂—NH), 32.24 (CH₂ alkyl chain), 30.68-29.19 (CH₂ alkyl chain), 27.35 (CH₂ alkyl chain), 22.82 (CH₂ alkyl chain), 13.84 (CH₃ alkyl chain).

### N-Hexadecyl-(D-Glucuronyl-(α-1,4)-D-Xyluronamide)Dibutylamide (Mal16diBu)

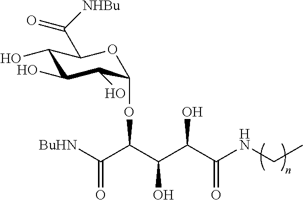

N-hexadecyl-D-glucuronyl-(α-1,4)-D-xyluronamide (0.127 mmol, 79 mg) and H₂SO₄/SiO₂ 22 wt % (0.305 mmol, 136 mg) are added to a zirconia bowl containing 80 zirconia balls with a diameter of 5 mL. MeOH (0.15 mL) is added. The medium is ground in a planetary ball mill (P7PL) at 500 rpm for 8 cycles of 5 min with a break of 2 min, in reverse mode. The methyl ester obtained is directly involved in the subsequent reaction under the same grinding conditions. 99.5% butylamine (0.381 mmol, 38 μL) is added to the bowl, followed by MeOH (0.15 mL). The medium is milled in a planetary ball mill (P7PL) at 500 rpm for 8 cycles of 5 min with a break of 2 min, in reverse mode. The crude reaction is dissolved in MeOH, filtered to remove H₂SO₄/SiO₂ and evaporated. The residue is purified by automated flash chromatography on a 15 g SiO₂ cartridge. The elution gradient is: EtOAc/MeOH: 100/0 to 80/20 in 30 min. After evaporation and freeze drying, the Mal16diBu compound is obtained in the form of a white powder, with a yield of 40% (35 mg).
M: 689.93 g·mol⁻¹
Formula: C₃₅H₆₇N₃O₁₀
Yield: 0.57 (EtOAc/MeOH: 9/1)

¹H NMR (600 MHz, pyridine-d₅): 8.34 (m, 3H, NH), 5.70 (d, 1H, J=3.2 Hz, H_{Glc1′}), 5.22 (d, 1H, J=5.1 Hz, H_{Glc2}), 5.03 (m, 8H, H_{Glc3}, H_{Glc4}, H_{Glc5′} and OH), 4.59 (t, 1H, J=9.1 Hz, H_{Glc3′}), 4.27 (t, 1H, J=9.4 Hz, H_{Glc4′}), 4.13 (dd, 1H, J=9.5 Hz, J=3.4 Hz, H_{Glc2′}), 3.45 (m, 5H, CH₂—NH), 3.27 (s^{x}, 1H, J=6.5 Hz, CH₂—NH), 1.53 (m, 6H, CH₂—CH₂—NH), 1.24 (m, 30H, CH₂ hexadecylamine and butylamine chains), 0.86 (t, 3H, J=6.1 Hz, CH₃ hexadecylamine and butylamine chains), 0.79 (m, 6H, CH₃ hexadecylamine and butylamine chains)

¹³C NMR (150 MHz, pyridine-d₅): 173.53 (C_{Glc1}), 171.75 (C_{Glc5} or C_{Glc6′}), 171.71 (C_{Glc5} or C_{Glc6′}), 103.19 (C_{Glc1′}), 84.74 (C_{Glc4}), 74.61 (C_{Glc3′}), 74.16 (C_{Glc4′}), 74.10 (C_{Glc2}), 73.91 (C_{Glc3} or C_{Glc5′}), 73.22 (C_{Glc2′}), 39.85 (CH₂—NH butyl chain), 39.68 (CH₂—NH butyl chain), 39.47 (CH₂—NH hexadecyl chain), 32.09 (CH₂ hexadecyl chain), 32.38 (CH₂ butyl chain), 32.36 (CH₂ butyl chain), 30.58-29.98 (CH₂ hexadecyl chain), 27.66 (CH₂ hexadecyl chain), 23.31 (CH₂ hexadecyl chain), 20.79 (CH₂ butyl chain), 20.69 (CH₂ butyl chain), 14.65 (CH₃ hexadecyl chain), 14.26 (CH₃ butyl chain), 14.24 (CH₃ butyl chain)

N-hexadecyl-(D-glucuronyl-(α-1,4)-D-xyluronamide)dibutylamide persulphate (Mal16diBu-perS)

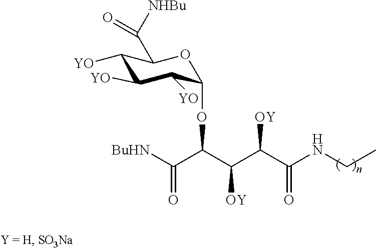

The Mal16diBu-perS derivative is obtained following the same procedure described for obtaining Mal16perS. The Mal16diBu compound (35 mg, 0.051 mmol) and 16 equivalents of 97% SO₃/active pyridine are introduced into a 20 mL zirconia bowl containing 80 zirconia balls with a diameter of 5 mm, then into the P7PL Fritsch planetary mill. The mixture is ground at 400 rpm for 48 cycles of 5 min. Then 16 equivalents of NaHCO₃ (69 mg, 0.81 mmol) are introduced into the bowl and the mixture is ground again for 6 cycles of 5 minutes. The crude reaction is then dissolved in milliQ water and dialyzed using a 1000 kD cutoff membrane (3×500 mL). Mal16diBu-perS (52 mg) is obtained in the form of mixtures of N-hexadecyl-(D-glucuronyl-(α-1,4)-D-xyluronamide)dibutylamide tri, tetra, and penta sulphates.

### III. Biological Data

### 1) In Vitro Tests for Abnormal Phosphorylation of Tau

Phosphorylation of tau protein is the most common post-translational modification to regulate tau activity and functions. The longest tau isoform (4R2N, 441 amino acids) containing 85 potential phosphorylation sites was used. In vitro phosphorylation of the tau protein is done by induction, on the one hand, (FIG. 5) and, on the other hand, according to a competition process between the LG2A molecules and heparin, conventionally used to induce abnormal tau phosphorylation in vitro (FIGS. 3, 4, 6, 7, 8, 9 and 10).

For abnormal tau phosphorylation tests, GSK3p, a kinase centrally involved in this process in the brains of subjects with Alzheimer's disease (AD), was used. Analysis of tau phosphorylation by GSK3b at sites characteristic of AD is possible by the western blot technique using specific antibodies that recognize abnormally phosphorylated sites in the brain or cerebrospinal fluid of AD subjects, such as the AT270 antibody which recognizes threonine 181 (Thr181), the PHF1 antibody which recognizes serines 396 and 404 (Ser396/Ser404), and the AT8 antibody, which recognizes the Ser199/Ser202/Thr205 site.

### Experimental Protocol

The composition of the reaction media is as follows:

By induction: Tau protein (40 μg/mL) (Millipore) is phosphorylated in the presence of ATP (Sigma-Aldrich) at 48 μM and protease inhibitors (Sigma-Aldrich, 1/100), by the GSK3β enzyme at 4 ng/mL (Promega), in buffer (40 mM Tris [pH 7.5], 20 mM MgCl₂, 0.1 mg/mL BSA and 0.1 mM DTT). The LG2A molecules are added at 12 μg/mL and their effects are compared to those observed by the addition of the same concentration of heparin.

By competition: The tau protein (40 μg/mL) is phosphorylated by the GSK3β enzyme at 4 ng/mL (Promega) in the presence of ATP (Sigma-Aldrich) at 48 μM and protease inhibitors (Sigma-Aldrich, 1/100), in the buffer mentioned above. The ability of the LG2A molecules (60 μg/mL) to compete with heparin (12 μg/mL; Sigma-Aldrich) is evaluated. Thus if the signal in the presence of the molecules is less than that obtained with heparin alone, the molecules are then defined as “protectors” from abnormal tau phosphorylation.

In both systems (induction or competition), the tau protein is incubated at 37° C. with stirring in the presence of the different elements presented above. The reaction kinetics are monitored by taking 8 μL of the samples at 1.5 h, 3 h and 6 h of incubation. Loading buffer (Euromedex) and β-mercaptoethanol (Sigma-Aldrich) are added to each sample. The samples are then stored at −20° C. until analysis by western blot.

After denaturation of the samples by heating at 95° C. for 5 minutes, the samples are deposited simultaneously with the molecular weight marker (Precision Plus Protein™ Dual Color Standards; BIO-RAD) on a 10% acrylamide gel placed in an electrophoresis tank containing migration buffer (0.25 M Tris; 1.6 M Glycine; 20% SDS; pH 8.6).

The controls used for this phosphorylation experiment followed by western blot are:
the “native tau” protein, that is to say the non-phosphorylated tau protein.
the tau protein phosphorylated by GSK3b alone in the absence of heparin.
the tau protein phosphorylated in the presence of heparin, denoted “tau+heparin”.
the “external standard” corresponding to the tau protein phosphorylated in the presence of heparin for 3 h. This standard is carried out in an experiment independently of the phosphorylation experiments in the presence of LG2A compounds. It ensures that the western blot is carried out correctly.

The migration takes place at constant 80 V until the arrival of the proteins in the separation gel visualized by the migration front of bromophenol blue contained in the loading buffer, then at constant 120 V until the arrival of the migration front to the other end of the gel.

At the end of the migration, a semi-dry transfer is carried out on a polyvinylidene fluoride (PVDF) membrane using the “Trans-blot Turbo RTA Transfer Kit, PVDF” (Bio-Rad). The PVDF membrane is activated in methanol (VWR) beforehand and then placed in the transfer buffer provided in the kit.

The transfer is carried out at 25 V for 10 minutes.

Once the transfer of proteins onto the PVDF membrane is completed, a saturation step is carried out in PBS with 3% skimmed milk powder (Merck Millipore) and 0.1% Tween 20 (VWR) for 1 h with stirring at room temperature.

Then the membrane is incubated overnight with stirring at +4° C. in a solution of PBS, 0.1% Tween and 1% milk containing the primary antibody specific for tau phosphorylation sites (AT270, ThermoScientific or S199-202, Millipore).

After incubation of the PVDF membrane in the chosen primary antibody, a succession of 3 washes of 5 minutes of the membrane is carried out in PBS and 0.1% Tween with stirring and the membrane is incubated for 1 h with stirring at room temperature in a solution of 1×PBS, 0.1% Tween and 1% milk containing the diluted secondary antibody (Jackson Immuno-Research). After 3 washes of 5 minutes with PBS and 0.1% Tween, then one wash with PBS, the membrane is revealed using the “Immobilon Forte Western HRP Substrate” kit (Merck Millipore) and the signal recorded by the LI-COR device (Odyssey).

### Results

In the absence of heparin (highly sulphated HS), no phosphorylation of tau on sites characteristic of AD is observable (Sepulveda-Diaz et al, 2015) (FIG. 2). However, in the presence of heparin (highly sulphated HS) phosphorylation at all tau sites takes place (p-tau), reproducing the process of abnormal hyperphosphorylation that takes place in the cell before the establishment of tauopathy.

Using the in vitro test, it has been demonstrated that the synthesized disulphated C16 maltobionamides (Mal16diS) enter into total competition with heparin (hep), preventing heparin from inducing the phosphorylation of tau on the two abnormal tau phosphorylation sites characteristic of Alzheimer's disease analysed (AT270 and Ser 199/202) starting at 1.5 h and up to 6 h of reaction. Thus, the abnormal phosphorylation of tau (p-tau), revealed by the antibodies characteristic of Alzheimer's disease (AT270 and Ser199/202), is inhibited by the presence of disulphated C16 maltobionamides (Mal16diS) after 3 h of reaction. in vitro. The results are shown in FIG. 3.

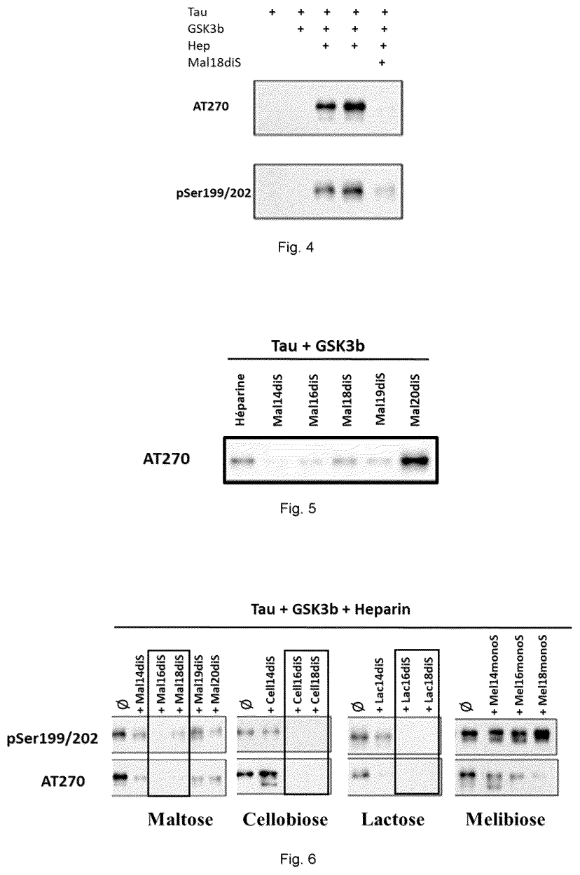

Likewise, the synthesized disulphated C18 maltobionamides (Mal18diS) enter into total competition with heparin (Hep) on the tau AD sites (AT270) after 1.5, 3 and 6 h of reaction. Thus, the abnormal phosphorylation of tau (p-tau), revealed by the antibodies characteristic of Alzheimer's disease (AT270 and pSer199/202) is inhibited by the presence of disulphated C18 maltobionamides (Mal18diS) after 3 h of reaction (FIG. 4).

However, when the capacity of maltobionamide compounds, with a hydrocarbon chain (R₁) comprising from 14 to 20 carbons (Mal14diS to Mal20diS) to themselves induce the abnormal phosphorylation of tau was studied, i.e., in the absence of heparin, it was demonstrated that the longer the hydrocarbon chains of the compounds are, the more they induce p-tau (FIG. 5).

Thus, for the same ability to protect tau from its abnormal phosphorylation, it is preferable to choose the compound with the shortest lipid chain length.

### Influence of Disaccharide

The analogous disulphated cellobionamide and lactobionamide derivatives with a hydrocarbon chain comprising 14 to 18 carbons were synthesized in order to study the influence of the nature of the disaccharide.

Only the cello- and lactobionamide compounds comprising a 16- and 18-carbon hydrocarbon chain, respectively “Cell16diS”, “Cell18diS”, “Lac16diS” and “Lac18diS”, show a result similar to the disulphated maltobionamides comprising a 16-carbon hydrocarbon chain (“Mal16diS”) and an 18-carbon hydrocarbon chain (“Mal18diS”), on their ability to enter into total competition with heparin from 1.5 to 6 h of tau phosphorylation by GSK3b, as shown in FIG. 6 for 3 h of reaction. The melibionamide derivatives comprising a 14, 16 or 18 carbon hydrocarbon chain respectively “Mel14monoS”, “Mel16monoS and “Mel18monoS) do not compete with heparin for abnormal tau phosphorylation (FIG. 6). Thus, by these latter derivatives, it appears that a single anionic charge or the conformation of the disaccharide is not favourable to compete with heparin and prevent abnormal phosphorylation of tau.

### Influence of Sulphation

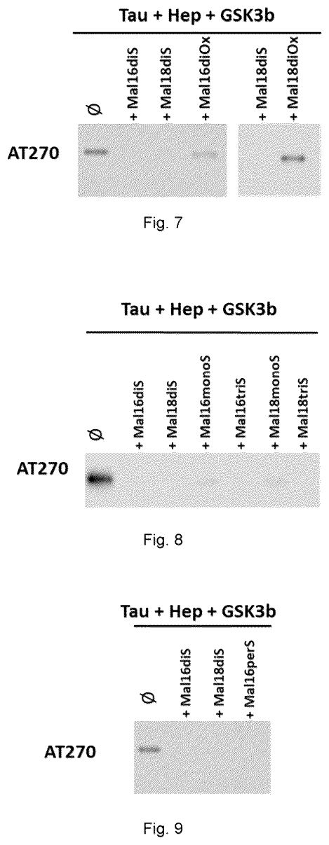

Oxidized compounds analogous to maltobionamides comprising a 16 or 18 carbon hydrocarbon chain were synthesized in order to estimate the necessity of sulphation of the saccharide part of the compounds for their anti-p-tau activity. It was observed that oxidized compounds comprising a 16-carbon hydrocarbon chain (“Mal16diOx”) were less effective than disulphated maltobionamide compounds with the same chain length (Mal16diS) in preventing tau phosphorylation (FIG. 7). The oxidized compound comprising an 18-carbon hydrocarbon chain (“Mal18diOx”) does not compete with heparin, unlike its disulphated analogue comprising an 18-carbon hydrocarbon chain (“Mal18diS”).

Thus the sulphation of the disaccharide is essential to compete with heparin (prototype of hypersulphated HS present in Alzheimer's disease) and prevent abnormal phosphorylation of tau.

To refine the importance of disaccharide sulphation, maltobionamide derivatives comprising a hydrocarbon chain with 16 or 18 monosulphated carbons respectively “mal16monoS” and “mal18monoS” or tri-sulphated carbons, respectively “mal16triS” and “mal18triS” were synthesized.

The compounds “Mal16monoS”, “Mal16triS”, “mal18monoS” and “mal18triS” showed (i) a slight loss of the protective capacity of tau against its heparin-induced phosphorylation when maltobionamide loses a sulphate (“Mal16monoS»and “Mal18 monoS”) and (ii) a maintenance of this capacity to protect tau when the compound gains a sulphate (“Mal16triS” and “Mal18triS” (FIG. 8).

Furthermore, when the 16-carbon maltobionamide compound is persulphated (Mal16perS), it retains its ability to protect tau from its abnormal phosphorylation induced by heparin like its disulphated homologue at the same lipid chain length (FIG. 9).

### 2) Induction of Tau Protein Aggregation

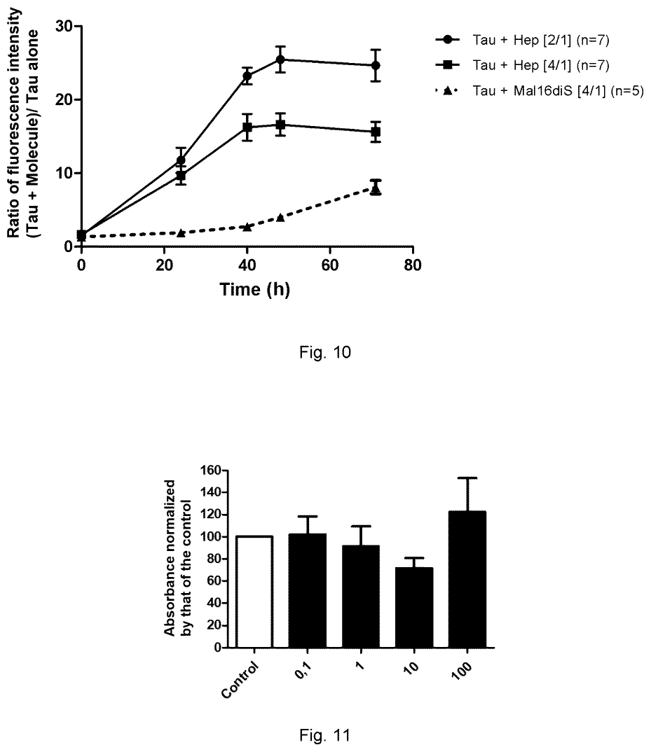

The ability of the 16-carbon disulphated maltobionamide compound “Mal16diS” to induce tau protein aggregation, another step in the occurrence of neurofibrillary tangles, was evaluated. Thus, the ability of this molecule to induce or inhibit the aggregation of tau in vitro (FIG. 10) was evaluated in a test described in the article by Sui et al, 2015 in which tau aggregation is monitored by the incorporation of Thioflavin T (ThT).

### Experimental Protocol

The aggregation test is carried out with tau protein (4R2N isoform, 441 amino acids) custom synthesized by the supplier Tebu-bio (manufactured on 17/04/19; initial conc.: 2.47 mg/mL) in a black 96-well plate with a final volume of 100 μL and under different conditions:
Tau protein (0.71 mg/mL) alone in buffer (40 mM Tris [pH 7.45], 200 mM NaCl, 2 mM EDTA and 1 mM DTT).
Tau protein (0.71 mg/mL) in the presence of heparin at 0.35 mg/mL (mass ratio of 2/1) or at 0.175 mg/mL (mass ratio of 4/1) in the buffer mentioned above.
Tau protein (0.71 mg/mL) in the presence of Mal16diS at 0.175 mg/mL (mass ratio of 4/1) in the buffer mentioned above.

100 μL of ThT (Sigma-Aldrich) at 50 μM is immediately added to each well, then the plate is incubated at 37° C. with stirring. Monitoring the formation of tau protein aggregates is possible by monitoring the incorporation of thioflavin leading to its fluorescence emission and is carried out by reading at 0, 24, 40, 48 and 71 h at Exc wavelength 450 nm/Em wavelength 510 nm, by means of an Infinite M1000 spectrofluorometer (Tecan).

### Results

The Mal16diS compound tested induces the aggregation of tau protein more weakly compared to heparin up to 72 h of reaction (FIG. 10).

3) In cellulo tests for abnormal phosphorylation of tau

Before testing tau phosphorylation in cellulo, a cytotoxicity study was carried out on the neuroblastoma cell line, SH-SY5Y.

### a) Cytotoxicity Study

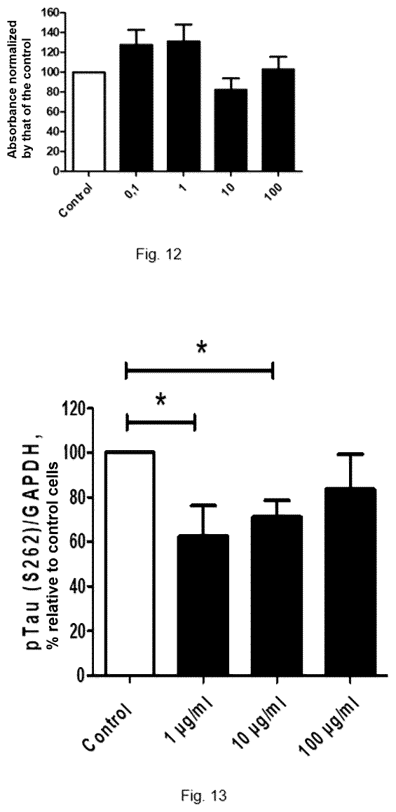

The cytotoxicity tests were carried out on the molecules which showed the best potential in the tau phosphorylation tests in vitro: disulphated maltobionamides with 16 carbons and 18 carbons. In order to evaluate the level of toxicity of these molecules according to their concentration in the cellular environment, an MTT test (3-(4,5-dimethylthiazol-2-yl)-2,5-diphenyl tetrazolium bromide) is carried out (FIGS. 11 and 12) upstream of the phosphorylation tests in cellulo.

### Experimental Protocol

SH-SY5Y (ATCC-CRL-2266) are developed in Dulbecco's Modified Eagle Complete Medium (DMEM-GlutaMAX™, Life Technologies) with 10% foetal bovine serum (FBS) and 1% penicillin/streptomycin in an incubator at 37° C. with 5% CO₂.

For this, the SH-SY5Y cells are cultured in a 6-well plate at a cell density of 200,000 cells/well and are differentiated using retinoic acid (10 μM, Sigma Aldrich) for 5 days. On day 6, the cells are brought into contact with LG2A molecules at different concentrations (0.1, 1, 10 or 100 μg/mL), in a final volume of 2 mL. After 24 hours, MTT (5 μg/mL; Sigma-Aldrich) is added to the cell medium for 2 hours at 37° C. Then the medium is removed and 200 μL of pure dimethylsulfoxide (DMSO, Sigma-Aldrich) are added to each well. The entire contents of each well are transferred to a 96-well plate and the absorbance is measured at 562 nm using the Infinite M1000 spectrofluorometer (Tecan).

### Results

The 16-carbon disulphated maltobionamides (Mal16diS) are not cytotoxic to SH-SY5Y from the lowest dose of 0.1 μg/mL to the highest dose of 100 μg/mL (FIG. 11).

Similarly, it has been shown that 18-carbon disulphated maltobionamides (Mal18diS) are not cytotoxic to SH-SY5Y from the lowest dose of 0.1 μg/mL to the highest dose of 100 μg/mL (FIG. 12).

Furthermore, the compounds tested were not seen to induce an additional inflammatory state, via interleukin 6 assay, of HMC3 cells after stimulation with lipopolysaccharide (LPS) (50 ng/mL) when the compounds are added at doses of 0.1, 1 or 10 μg/mL compared to the same cells receiving only LPS (data not shown).

### b) In Cellulo Test in the SH-SY5Y Line

The tau phosphorylation induction model on the SH-SY5Y cell line described in the Gly-CRRET laboratory article was used here (Sepulveda-Diaz et al., 2015).

### Experimental Protocol

In short, SH-SY5Y cells are seeded in 6-well plates and differentiated using retinoic acid (10 μM, Sigma Aldrich) for 5 days. On day 6, the cells are placed in the presence of the Mal16diS and Mal18diS molecules at different concentrations (1, 10 or 100 μg/mL), in a final volume of 2 mL simultaneously with a well not receiving molecules. After 24 hours, the molecules are removed and the stress inducer (H₂O₂, Sigma Aldrich) is added to the cell medium for 30 minutes. The cells are then rinsed with 1×PBS then lysed and recovered in RIPA buffer (Thermo Fisher). The protein extracts from the cells (10 μg) are then analysed by western blot according to the same procedure described above.

The tau phosphorylation site studied here is the S262 site (Thermo Fisher), normalized to the expression of the GAPDH protein (Sigma Aldrich). The bands are quantified using the Odyssey software of the LI-COR device allowing the p-tau/GAPDH ratio to be calculated for each of the conditions. The ratio obtained for each concentration of molecule is then expressed as a percentage of the ratio obtained for the control cells, i.e., not treated by the molecules. Thus a value less than 100% corresponds to protection provided by the molecules in this cellular model of tau phosphorylation induction.

The experiment was repeated at least 4 times for each molecule dose for the “Mal16diS” and “Mal18diS” molecules and at least twice for the “Cell16diS” and “Lac16diS” molecules. The mean and standard deviation of the replicates are shown in FIGS. 13 to 16; each of the values obtained for each dose is compared to that of the control without addition of molecule by means of a t-test. Statistical differences with p-value <0.05 and <0.001 are represented by “*” and “***” respectively.

### Results

The two lowest doses of 16-carbon disulphated maltobionamides (“mal16diS”) tested (1 and 10 μg/mL) allow a significant reduction (p<0.01) of 30 and 40% in tau phosphorylation in SH-SY5Y cells compared to untreated cells (FIG. 13).

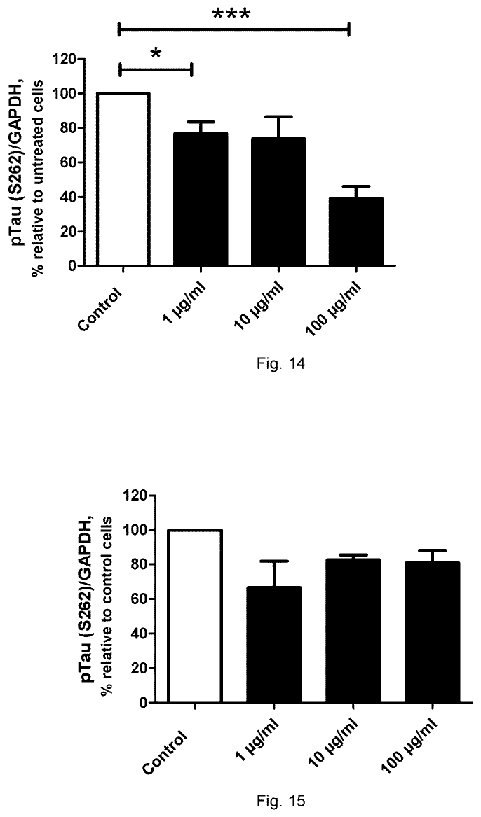

In 18-carbon disulphated maltobionamide (“mal18diS”), a greater decrease in tau phosphorylation is observed with increasing concentration of the compound. With the highest dose of 100 μg/mL, up to 60% reduction in tau phosphorylation was observed compared to untreated cells (p<0.001); the lowest doses provide protection from tau phosphorylation induced by oxidative stress close to 30% (FIG. 14).

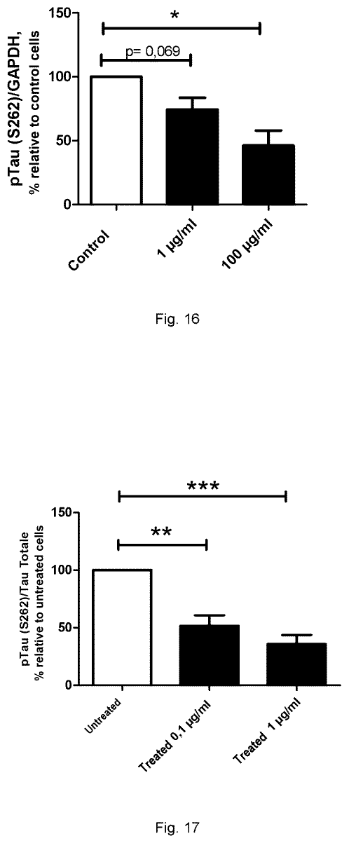

For the lowest dose of 1 μg/mL, 16-carbon disulphated cellobionamide (“cell16diS”) provides an approximately 34% decrease in tau phosphorylation in SH-SY5Y cells compared to untreated cells; however, this difference is not statistically different. It is the same for doses of 10 and 100 μg/mL of this compound where the protection of tau is 17 and 20% respectively (FIG. 15). Finally, for 16-carbon disulphated lactobionamide (“lac16diS”), the lowest dose of 1 μg/mL provides an almost significant (p=0.069) 25% decrease in tau phosphorylation in SH-SY5Y cells. The 10 μg/mL dose of this compound has not been tested. The highest dose of 100 μg/mL of this molecule provides protection from tau protein phosphorylation of 54% (p<0.01) compared to untreated control cells (FIG. 16).

c) In Cellulo Test in Cortical Cells of rTg4510 Mice

Cortical cells from rTg4510 mice mutated for human tau protein (P301 L) were used as a model of primary cells expressing a tauopathy without induction by an effector, unlike the previous cellular model (Santa Cruz et al., Science, 2005).

### Experimental Protocol

Cortical cells are extracted from mouse embryos expressing the P301L mutation at stage E16 and cultured in a 6-well plate. On day 14 (moderate tau phosphorylation) or day 18 (severe tau phosphorylation), cells extracted from the same embryo are placed in the presence of the Mal16diS molecule at different concentrations (0.1 or 1 μg/mL), in a final volume of 2 mL simultaneously with a well of cells from the same embryo not receiving molecules for 24 h. After 24 h of treatment with the molecules, the cells are rinsed with 1×PBS then lysed and recovered in RIPA buffer (Thermo Fischer). The protein extracts from the cells (10 μg) are then analysed by western blot according to the same procedure described above.

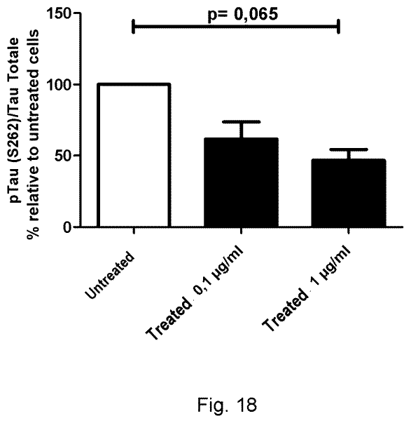

The tau phosphorylation site studied here is the S262 site (Thermo Fisher), normalized to the expression of total tau protein (K9JA, Dako). The bands are quantified using the Odyssey software of the LI-COR device allowing the p-tau/tau ratio to be calculated for each of the conditions. The ratio obtained for each concentration of molecule is then expressed as a percentage of the ratio obtained for cells not treated by the molecules. Thus a value less than 100% corresponds to protection provided by the molecules in this cellular model of expression of tau phosphorylation. The experiment was repeated at least three times for each dose of “Mal16diS” molecule. The mean and standard deviation of the replicates are shown in FIGS. 17 and 18; each of the values obtained for each dose is compared to that of the cells without addition of the molecule, with a t-test. Statistical differences with p-value <0.01 and <0.001 are represented by “**” and “***” respectively.

### Results

Both doses of 16-carbon disulphated maltobionamide tested (“mal16diS”) (0.1 and 1 μg/mL) allow a significant reduction of 48% (p<0.01) and 64% (p<0.001) in tau phosphorylation when this phosphorylation is moderate (D14) in the cortical cells of rTg4510 mice compared to untreated cells (FIG. 17, n=4).

This reduction in phosphorylation in primary cultures of cortical cells is maintained when the cells exhibit more severe phosphorylation (D18) before treatment, notably with a reduction of 39% and 53% for doses of 0.1 and 1 μg/mL, respectively, of “mal16diS” (FIG. 18, n=4).

1-11. (canceled)

12. A pharmaceutical composition comprising a compound of formula (I): 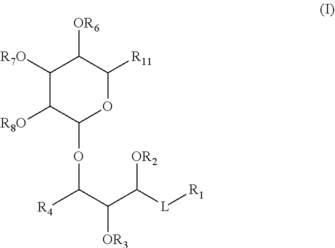
in which:
L is a —C(═O)NH—, —C(═O)O— or —C(═O)S— group
R₁ is a linear or branched, saturated or unsaturated hydrocarbon chain, comprising 15, 16, 17 or 18 carbon atoms, optionally substituted in the terminal position by a group chosen from —OH, —OR, —NH₂, —NHR, —NRR′, —COOH, —COOR, —CONHR, —CONRR′, and —SR, with R and R′ independently designating an alkyl group comprising from one to six carbon atoms;
R₄ is a group of formula (IIa) or (IIb): 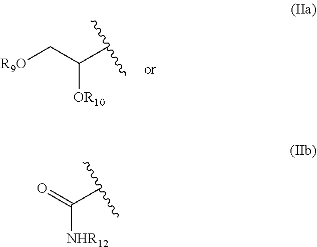
in which:
R₉ and R₁₀ are, independently of each other, a hydrogen atom or a sulphonate group in ionized form;
R₁₂ is an alkyl group comprising one to six carbon atoms;
R₁₁ is a —CH₂—OR₅ or —CONHR₁₃ group in which:
R₅ is a hydrogen atom or a sulphonate group in ionized form;
R₁₃ is an alkyl group comprising one to six carbon atoms:
R₂, R₃, R₆, R₇ and R₈ are, independently of each other, a hydrogen atom or a sulphonate group in ionized form; and
in which at least one of the R₂, R₃, R₅, R₇, R₈, R₉ and R₁₀ groups are a sulphonate group in ionized form;
and one or more pharmaceutically acceptable excipients.

13. (canceled)

14. A compound having the following structure (Ic): 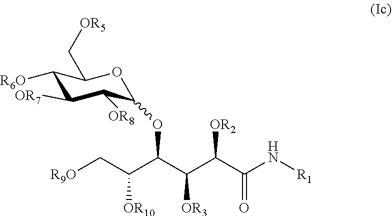
R₁ is a linear or branched, saturated or unsaturated hydrocarbon chain, comprising 15, 16, 17 or 18 carbon atoms, optionally substituted in the terminal position by a group chosen from —OH, —OR, —NH₂, —NHR, —NRR′, —COOH, —COOR, —CONHR, —CONRR′, and —SR, with R and R′ independently designating an alkyl group comprising from one to six carbon atoms;
R₅ is a hydrogen atom or a sulphonate group in ionized form;
R₉ and R₁₀ are, independently of each other, a hydrogen atom or a sulphonate group in ionized form;
R₂, R₃, R₆, R₇ and R₈ are, independently of each other, a hydrogen atom or a sulphonate group in ionized form; and
in which at least one of the R₂, R₃, R₅, R₆, R₇, R₈, R₉ and R₁₀ groups, preferably at least two of the R₂, R₃, R₅, R₆, R₇, R₈, R₉ and R₁₀ groups are a sulphonate group in ionized form.

15. The pharmaceutical composition according to claim 12, wherein, in the compound of formula (I), Rn is a CH₂—OR₅ group.

16. The pharmaceutical composition according to claim 12, wherein, the compound of formula (I) has the following formula (Ib):

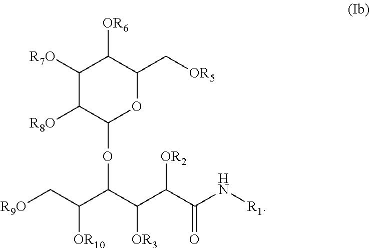

17. The pharmaceutical composition according to claim 12, wherein the compound of formula (I) has the following formula (Ic):

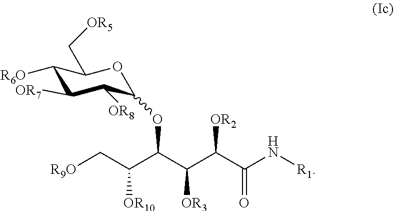

18. The pharmaceutical composition according to claim 12, wherein, the compound of formula (I) has the following structure (Ie):

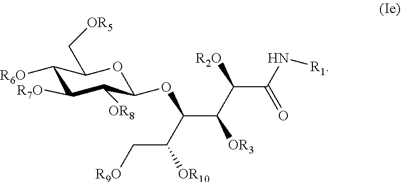

19. The pharmaceutical composition according to claim 12, wherein, the compound of formula (I) has the following structure (Ig):

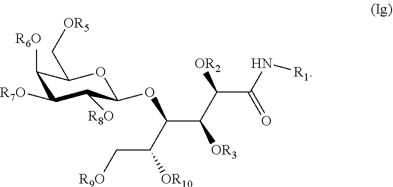

20. The pharmaceutical composition according to claim 12, wherein, in the compound of formula (I), R₁ is a saturated linear hydrocarbon chain comprising 15, 16, 17 or 18 carbon atoms.

21. The pharmaceutical composition according to claim 12 wherein, in the compound of formula (I), R₂, R₃, R₆, R₇, R₈ and R₁₀ are hydrogen atoms and R₅ and R₉ are sulphonate groups in ionized form.

22. The pharmaceutical composition according to claim 12, wherein, in the compound of formula (I), R₁ is a saturated linear hydrocarbon chain comprising 16 or 18 carbon atoms.

23. The pharmaceutical composition according to claim 12, wherein, in the compound of formula (I), at least two of the R₂, R₃, R₅, R₆, R₇, R₈, R₉ and R₁₀ groups are a sulphonate group in ionized form.

24. The pharmaceutical composition according to claim 12, said compound being chosen from the following compounds:

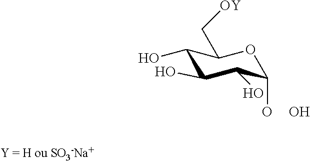

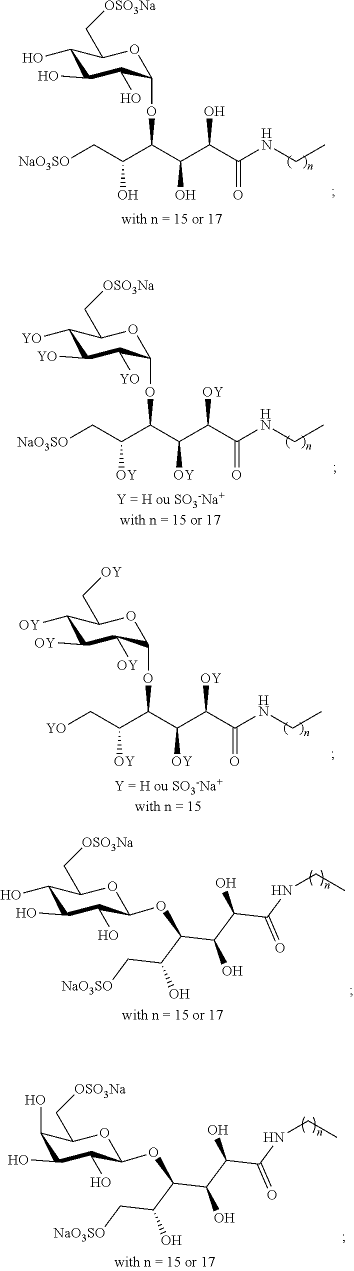

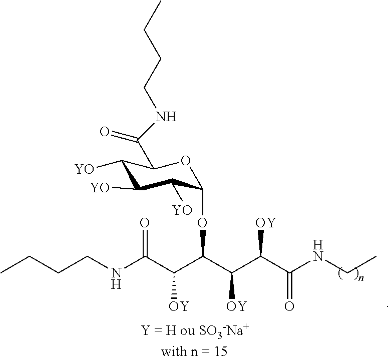

25. A method for treating a tauopathy comprising the administration, to an individual in need, of an effective dose of a compound of formula (I) 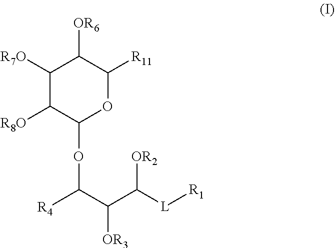
in which:
L is a —C(═O)NH—, —C(═O)O— or —C(═O)S— group
R₁ is a linear or branched, saturated or unsaturated hydrocarbon chain, comprising 15, 16, 17 or 18 carbon atoms, optionally substituted in the terminal position by a group chosen from —OH, —OR, —NH₂, —NHR, —NRR′, —COOH, —COOR, —CONHR, —CONRR′, and —SR, with R and R′ independently designating an alkyl group comprising from one to six carbon atoms;
R₄ is a group of formula (IIa) or (IIb): 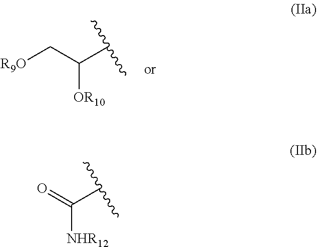
in which:
R₉ and R₁₀ are, independently of each other, a hydrogen atom or a sulphonate group in ionized form;
R₁₂ is an alkyl group comprising one to six carbon atoms;
R₁₁ is a —CH₂—OR₅ or —CONHR₁₃ group in which:
R₅ is a hydrogen atom or a sulphonate group in ionized form;
R₁₃ is an alkyl group comprising one to six carbon atoms;
R₂, R₃, R₆, R₇ and R₈ are, independently of each other, a hydrogen atom or a sulphonate group in ionized form; and
in which at least one of the R₂, R₃, R₅, R₆, R₇, R₈, R₉ and R₁₀ groups are a sulphonate group in ionized form.

26. The method according to claim 25, wherein, in the compound of formula (I), R₁₁ is a CH₂—OR₅ group.

27. The method according to claim 25, wherein, the compound of formula (I) has the following formula (Ib):

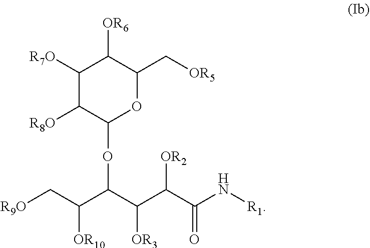

28. The method according to claim 25, wherein the compound of formula (I) has the following formula (Ic):

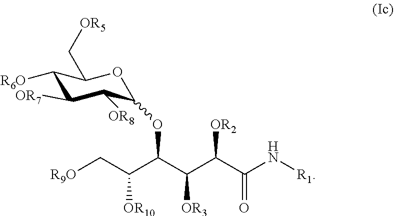

29. The method according to claim 25, wherein, the compound of formula (I) has the following structure (Ie):

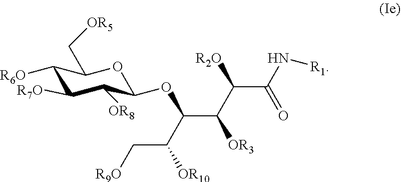

30. The method according to claim 25, wherein, the compound of formula (I) has the following structure (Ig):

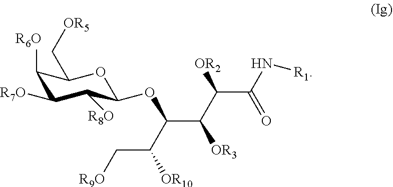

31. The method according to claim 25, wherein, in the compound of formula (I), R₁ is a saturated linear hydrocarbon chain comprising 15, 16, 17 or 18 carbon atoms.

32. The method according to claim 25, wherein, in the compound of formula (I), R₂, R₃, R₆, R₇, R₈ and R₁₀ are hydrogen atoms and R₅ and R₉ are sulphonate groups in ionized form.

33. The method according to claim 25, wherein, in the compound of formula (I), R₁ is a saturated linear hydrocarbon chain comprising 16 or 18 carbon atoms.

34. The method according to claim 25, wherein, in the compound of formula (I), at least two of the R₂, R₃, R₅, R₆, R₇, R₈, R₉ and R₁₀ groups are a sulphonate group in ionized form.

35. The method according to claim 25, said compound being chosen from the following compounds:

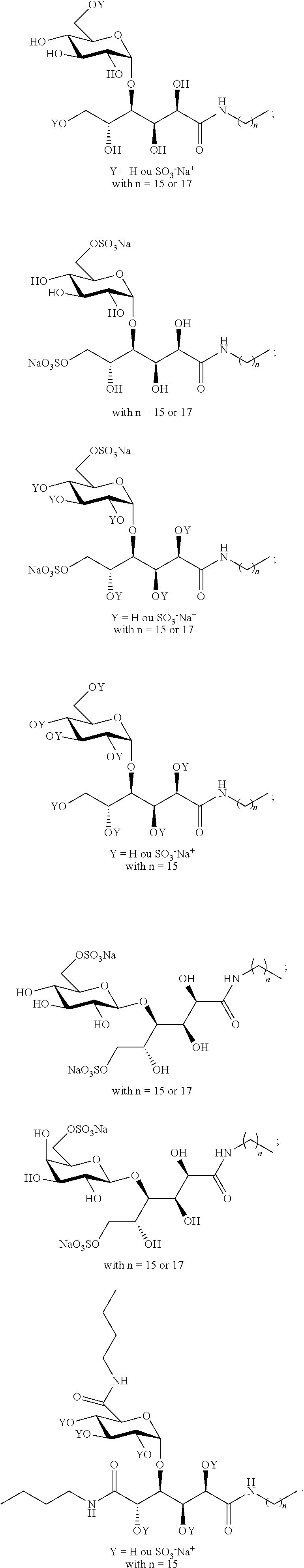

36. The method according to claim 25, wherein the tauopathy is Alzheimer's disease.
